SEQUENCE LISTING (46 sequences)

SEQ ID NO: 1 - DNA, 2274 bp, Arabidopsis thaliana
  source 1..2274: mol_type = unassigned DNA
gaatctctccctctttcttccatttcccagaactctcagagtttcgccacttcttctgaagaatctgaaagatcacaaagtccaaagaaaaaaaatgagcgtgatcacaactccaatagagaccctgcacctaaagtcaacacttcgtcttctaccacgagctgtttatcgcagtcaacggattcaagttttcccgcccaacatcttctcgaacacttctctgagttctccacttcgcatcgacccaatctctcaaggtttgttcatcttactctcatcgatcctcaggcgtttatgcttgttgggtacctgcaaaatttacactttcttctctcatggttttcttcaacctactatatacacagttggcggatcgaggaatttgtggcgtagatatgcatctgataatttctcggagatgggtttggatcctggagctgatccattcaaggtgtgcgctatgggatctttccttttccattctatgttatgttgggtttgaatagtcttctcaaaatgtgtcatctttaatgtcaaaatgtgattttgcactattgaagacttgttaattatcctacttttgaatctgattagctaaaaatcatccctggatcacagttttgctactttgttcttcgtttgctgatgatttcagttgttaagaattttatgttacttccgagaaccaacatgaatctagtaagtggcttgcaggttattgagaagccttctatagtggatagaatgaagaaagcaaactcaattcttcctcatgtagtgttggcgagtacaatattagctcttatctatcctccttctttcacttggtttacgtcgaggttcttcccattggaacaaggtcaaattgatctaagactttgaattgtagacatgaggtttgctaaaaagctcttctttactctcctgttattttaggtactttgtgcccgctttaggttttttgatgtttgcggttggtatcaattcaaacgagaaagactttcttgaagctttcaaaagaccaaaagctatcctcctcggttatgttggacaatatctcgtaaagcctgtcctaggtttcatctttggcctagctgctgtttctcttttccaactcccaactccaattggtaaaagctccctgtctctcatcattcaagactcttaacctatctctcacattaaccaatctttctcaatctctcaggtgctggaatcatgttggtatcatgtgttagtggagctcagttgtcaaactatgcgacattcctaactgatccagcattggcacctcttagcatcgtcatgacatctctatcaaccgctactgcggttcttgtcacaccgatgctatcactcctgctcattgggaagaaactacccgttgatgtaaaaggaatgatatccagcattcttcaggttgtaatcgcaccaatcgctgcaggattgctactaaacaagtgagagatatagctacaagcctacaactagaaataaccacaagttgtttctctaagtataaccatttttatctcctttttgtttcttcttttacaggttgttcccaaaagtatcaaatgcaatccgaccatttctcccgattctatcggttctcgacacagcttgctgcgttggagcaccactcgcattgaacataaactcggtcatgtctccatttggagccaccatattgttactagtaacaatgtttcatctctcagctttcctcgctggatacttccttaccggttctgtctttagaaacgctccagacgctaaagccatgcaaagaacattgtcctatgaaactggtactaactctttatggtattggaaaatcttatcaagaaagtacaatttggtttaaagatttgagaagagaaatgtgtttgtgaatatatcaggaatgcagagtagccttttggctctagcgcttgctactaagttctttcaagatcctcttgtggggattcctcctgctatatctgtaagtctcatatccttttgacatagcataatcttttattgatgatgattatagagatttttgttttgttttgcagacggtggtaatgtcattgatggggttcactctcgttttgatctggtctaaggaaaagagtaacacattttaacattttgattttgtttcactccacattttgattatacatatatatatgtactacaaataattggaaaattcttcttgtaatataaataattcaatacagaatttgagatttttaatgtactaatctccattttgtcttt

SEQ ID NO: 2 - DNA, 1463 bp, Arabidopsis thaliana
  source 1..1463: mol_type = unassigned DNA
gaatctctccctctttcttccatttcccagaactctcagagtttcgccacttcttctgaagaatctgaaagatcacaaagtccaaagaaaaaaaatgagcgtgatcacaactccaatagagaccctgcacctaaagtcaacacttcgtcttctaccacgagctgtttatcgcagtcaacggattcaagttttcccgcccaacatcttctcgaacacttctctgagttctccacttcgcatcgacccaatctctcaagttggcggatcgaggaatttgtggcgtagatatgcatctgataatttctcggagatgggtttggatcctggagctgatccattcaaggttattgagaagccttctatagtggatagaatgaagaaagcaaactcaattcttcctcatgtagtgttggcgagtacaatattagctcttatctatcctccttctttcacttggtttacgtcgaggtactttgtgcccgctttaggttttttgatgtttgcggttggtatcaattcaaacgagaaagactttcttgaagctttcaaaagaccaaaagctatcctcctcggttatgttggacaatatctcgtaaagcctgtcctaggtttcatctttggcctagctgctgtttctcttttccaactcccaactccaattggtgctggaatcatgttggtatcatgtgttagtggagctcagttgtcaaactatgcgacattcctaactgatccagcattggcacctcttagcatcgtcatgacatctctatcaaccgctactgcggttcttgtcacaccgatgctatcactcctgctcattgggaagaaactacccgttgatgtaaaaggaatgatatccagcattcttcaggttgtaatcgcaccaatcgctgcaggattgctactaaacaagttgttcccaaaagtatcaaatgcaatccgaccatttctcccgattctatcggttctcgacacagcttgctgcgttggagcaccactcgcattgaacataaactcggtcatgtctccatttggagccaccatattgttactagtaacaatgtttcatctctcagctttcctcgctggatacttccttaccggttctgtctttagaaacgctccagacgctaaagccatgcaaagaacattgtcctatgaaactggaatgcagagtagccttttggctctagcgcttgctactaagttctttcaagatcctcttgtggggattcctcctgctatatctacggtggtaatgtcattgatggggttcactctcgttttgatctggtctaaggaaaagagtaacacattttaacattttgattttgtttcactccacattttgattatacatatatatatgtactacaaataattggaaaattcttcttgtaatataaataattcaatacagaatttgagatttttaatgtactaatctccattttgtcttt

SEQ ID NO: 3 - protein, 409 aa, Arabidopsis thaliana
MSVITTPIETLHLKSTLRLLPRAVYRSQRIQVFPPNIFSNTSLSSPLRIDPISQVGGSRNLWRRYASDNFSEMGLDPGADPFKVIEKPSIVDRMKKANSILPHVVLASTILALIYPPSFTWFTSRYFVPALGFLMFAVGINSNEKDFLEAFKRPKAILLGYVGQYLVKPVLGFIFGLAAVSLFQLPTPIGAGIMLVSCVSGAQLSNYATFLTDPALAPLSIVMTSLSTATAVLVTPMLSLLLIGKKLPVDVKGMISSILQVVIAPIAAGLLLNKLFPKVSNAIRPFLPILSVLDTACCVGAPLALNINSVMSPFGATILLLVTMFHLSAFLAGYFLTGSVFRNAPDAKAMQRTLSYETGMQSSLLALALATKFFQDPLVGIPPAISTVVMSLMGFTLVLIWSKEKSNTF

SEQ ID NO: 4 - DNA, 2459 bp, Arabidopsis thaliana
  source 1..2459: mol_type = unassigned DNA
gtgccgtctcttagaatcacatccacgtcgtcgtctccatacccatggctactcttttagccactcctatcttctctcctttagcttcttctccagcaaggaaccgtctttcttgctctaagatccgtttcggttccaaaaatgggaaaattctcaattctgatggtgcccagaagttgaatctctcaaaattccgtaaacccgatggccaaagatttctacaaatgggttcttctaaagagatgaactttgagagaaaactctcagtccaagctatggatggtgcaggaacaggaaacacatcaacgatctctcgtaacgtaagcttcacgatgttgtttatttaccattttattgtaacagctttttaaagttttgaatttttcgttcaggtaattgcgataagtcacttgttggtatcacttgggatcattcttgctgcagactatttcttgaagcaggcgtttgtagcagcgtctattaagttcccaagtgctttgtttgggatgttctgtattttctctgttcttatgatatttgattcggttgttcctgctgctgcaaatggtttgatgaatttcttcgagcctgcgtttctgtttatccaaagatggcttcctttgttctatgttccttctcttgttgttcttcctctttctgttagagatattccggctgcttcaggtgtcaaaatctgctacattgtaggtatgaacttgatcttcacttggttaatgatcttgtgttctctggtagcttttatgggttttttgtctaattgctctgttttgtttattatttaatgctgatctgatgtcaatgttgatttgaatgtttgtttgtatagccggtggatggttggcgtcactttgtgtagcagggtacacagctattgcagtgagaaaaatggtgaaaaccgaaatgacggaagccgagcctatggcaaaaccatcaccattttcaacacttgagctatggagttggagtggaatctttgttgtgtcgtttgttggtgctctgttttaccctaattcattggggacaagtgcaagaacttctctccctttccttctttcttcaactgtgctaggttacattgtaggttctgggtaatattcaaaaacccttctttgttctccatctttaagcctctctatctcgcggttattctcagtgtcttatctttctttcttccaggttgccatcttctattaagaaagttttccatccgataatctgctgcgcgctatctgcagtacttgctgctctagcttttgggtatgcttcaggatctggacttgatcctgttttaggtaaagttaccttcactatgaaagaaactatagagaaacccttttttgacctaaaacagaaagtaaaagcattttgttaactgctctttaatggtgacaggaaactaccttaccaaagtagcatcagatcctggtgctggtgacatcttaatgggttttcttggctctgtcattctctctttcgctttctccatgttcaaacaaagaaaggtaaaacaaacaacaaacttgttctgttttgctttagaaaactggtaacaatcgctttggtttgcctctgtcttcttcagctcgtgaagaggcacgcagctgagatcttcacatctgtgatagtttcaacggtattctcgctctactccactgctcttgttggacgtttagtcggtttagaaccttctttaacggtttcaatcctacctcgctgcatcacggttgcattggcccttagcattgtatcactctttgaaggtatatatgtttcttcttcttctcaacacattaaagaacatccctaacgaatttgttggacattttcagggaccaattcgtctcttacagcagctgtagtcgttgtgactggtctgattggagctaactttgtacaagttgttcttgacaaactgcgtttacgtgatccaattgctcggggaattgcaactgcttcaaggtgactccattacaaaagatctttcacattttgaattaaaattatgcattaatgccattaggatcaaaagacatgacctttctattttttatggacagtgctcatggacttggaacagcagctttgtcggctaaggagccagaggctcttcccttttgtgcaatagcttatgctcttaccggaatcttcggatcgttactgtgttctgttcctgccgtccgacagagtttgctagcggtcgtcggctgaggattgatgatgtggcgaaacacgattgatggtctcaaagctggcgagttacttaaaatatgagagcagtttgctttggatgtcaaataagcttgtattaatgaaaaagatgatggaaaatgtttatttattttattttatatatgtaaatgttgtgagatttgtacaacatacataaattttatataggagtaggatgtcccagctttccaaaaatttgtcacaaaggt

SEQ ID NO: 5 - DNA, 1813 bp, Arabidopsis thaliana
  source 1..1813: mol_type = unassigned DNA
gtgccgtctcttagaatcacatccacgtcgtcgtctccatacccatggctactcttttagccactcctatcttctctcctttagcttcttctccagcaaggaaccgtctttcttgctctaagatccgtttcggttccaaaaatgggaaaattctcaattctgatggtgcccagaagttgaatctctcaaaattccgtaaacccgatggccaaagatttctacaaatgggttcttctaaagagatgaactttgagagaaaactctcagtccaagctatggatggtgcaggaacaggaaacacatcaacgatctctcgtaacgtaattgcgataagtcacttgttggtatcacttgggatcattcttgctgcagactatttcttgaagcaggcgtttgtagcagcgtctattaagttcccaagtgctttgtttgggatgttctgtattttctctgttcttatgatatttgattcggttgttcctgctgctgcaaatggtttgatgaatttcttcgagcctgcgtttctgtttatccaaagatggcttcctttgttctatgttccttctcttgttgttcttcctctttctgttagagatattccggctgcttcaggtgtcaaaatctgctacattgtagccggtggatggttggcgtcactttgtgtagcagggtacacagctattgcagtgagaaaaatggtgaaaaccgaaatgacggaagccgagcctatggcaaaaccatcaccattttcaacacttgagctatggagttggagtggaatctttgttgtgtcgtttgttggtgctctgttttaccctaattcattggggacaagtgcaagaacttctctccctttccttctttcttcaactgtgctaggttacattgtaggttctgggttgccatcttctattaagaaagttttccatccgataatctgctgcgcgctatctgcagtacttgctgctctagcttttgggtatgcttcaggatctggacttgatcctgttttaggaaactaccttaccaaagtagcatcagatcctggtgctggtgacatcttaatgggttttcttggctctgtcattctctctttcgctttctccatgttcaaacaaagaaagctcgtgaagaggcacgcagctgagatcttcacatctgtgatagtttcaacggtattctcgctctactccactgctcttgttggacgtttagtcggtttagaaccttctttaacggtttcaatcctacctcgctgcatcacggttgcattggcccttagcattgtatcactctttgaagggaccaattcgtctcttacagcagctgtagtcgttgtgactggtctgattggagctaactttgtacaagttgttcttgacaaactgcgtttacgtgatccaattgctcggggaattgcaactgcttcaagtgctcatggacttggaacagcagctttgtcggctaaggagccagaggctcttcccttttgtgcaatagcttatgctcttaccggaatcttcggatcgttactgtgttctgttcctgccgtccgacagagtttgctagcggtcgtcggctgaggattgatgatgtggcgaaacacgattgatggtctcaaagctggcgagttacttaaaatatgagagcagtttgctttggatgtcaaataagcttgtattaatgaaaaagatgatggaaaatgtttatttattttattttatatatgtaaatgttgtgagatttgtacaacatacataaattttatataggagtaggatgtcccagctttccaaaaatttgtcacaaaggt

SEQ ID NO: 6 - protein, 512 aa, Arabidopsis thaliana
MATLLATPIFSPLASSPARNRLSCSKIRFGSKNGKILNSDGAQKLNLSKFRKPDGQRFLQMGSSKEMNFERKLSVQAMDGAGTGNTSTISRNVIAISHLLVSLGIILAADYFLKQAFVAASIKFPSALFGMFCIFSVLMIFDSVVPAAANGLMNFFEPAFLFIQRWLPLFYVPSLVVLPLSVRDIPAASGVKICYIVAGGWLASLCVAGYTAIAVRKMVKTEMTEAEPMAKPSPFSTLELWSWSGIFVVSFVGALFYPNSLGTSARTSLPFLLSSTVLGYIVGSGLPSSIKKVFHPIICCALSAVLAALAFGYASGSGLDPVLGNYLTKVASDPGAGDILMGFLGSVILSFAFSMFKQRKLVKRHAAEIFTSVIVSTVFSLYSTALVGRLVGLEPSLTVSILPRCITVALALSIVSLFEGTNSSLTAAVVVVTGLIGANFVQVVLDKLRLRDPIARGIATASSAHGLGTAALSAKEPEALPFCAIAYALTGIFGSLLCSVPAVRQSLLAVVG

SEQ ID NO: 7 - DNA, 18 bp, synthetic construct
  misc_feature 1..18: note = CHEMICALLY SYNTHESIZED
  source 1..18: mol_type = other DNA
ctctctgccgacagtggt

SEQ ID NO: 8 - DNA, 23 bp, synthetic construct
  misc_feature 1..23: note = CHEMICALLY SYNTHESIZED
  source 1..23: mol_type = other DNA
cttatatgctcaacacatgagcg

SEQ ID NO: 9 - DNA, 21 bp, synthetic construct
  misc_feature 1..21: note = CHEMICALLY SYNTHESIZED
  source 1..21: mol_type = other DNA
ataaccgcgagatagagaggc

SEQ ID NO: 10 - DNA, 21 bp, synthetic construct
  misc_feature 1..21: note = CHEMICALLY SYNTHESIZED
  source 1..21: mol_type = other DNA
cccatggctactcttttagcc

SEQ ID NO: 11 - DNA, 19 bp, synthetic construct
  misc_feature 1..19: note = CHEMICALLY SYNTHESIZED
  source 1..19: mol_type = other DNA
attttgccgatttcggaac

SEQ ID NO: 12 - DNA, 39 bp, synthetic construct
  misc_feature 1..39: note = CHEMICALLY SYNTHESIZED
  source 1..39: mol_type = other DNA
gccggatccatggcttcgtgctctaagatccgtttcggt

SEQ ID NO: 13 - DNA, 28 bp, synthetic construct
  misc_feature 1..28: note = CHEMICALLY SYNTHESIZED
  source 1..28: mol_type = other DNA
gccctcgagtcagccgacgaccgctagc

SEQ ID NO: 14 - DNA, 28 bp, synthetic construct
  misc_feature 1..28: note = CHEMICALLY SYNTHESIZED
  source 1..28: mol_type = other DNA
gctctagaatgagcgtgatcacaactcc

SEQ ID NO: 15 - DNA, 29 bp, synthetic construct
  misc_feature 1..29: note = CHEMICALLY SYNTHESIZED
  source 1..29: mol_type = other DNA
gactcgagttaaaatgtgttactcttttc

SEQ ID NO: 16 - DNA, 26 bp, synthetic construct
  misc_feature 1..26: note = CHEMICALLY SYNTHESIZED
  source 1..26: mol_type = other DNA
ctactcttttagccactcctatcttc

SEQ ID NO: 17 - DNA, 20 bp, synthetic construct
  misc_feature 1..20: note = CHEMICALLY SYNTHESIZED
  source 1..20: mol_type = other DNA
agattcaacttctgggcacc

SEQ ID NO: 18 - DNA, 20 bp, synthetic construct
  misc_feature 1..20: note = CHEMICALLY SYNTHESIZED
  source 1..20: mol_type = other DNA
tcgcagtcaacggattcaag

SEQ ID NO: 19 - DNA, 20 bp, synthetic construct
  misc_feature 1..20: note = CHEMICALLY SYNTHESIZED
  source 1..20: mol_type = other DNA
tctacgccacaaattcctcg

SEQ ID NO: 20 - DNA, 20 bp, synthetic construct
  misc_feature 1..20: note = CHEMICALLY SYNTHESIZED
  source 1..20: mol_type = other DNA
tgagttcaacgagtgtgctg

SEQ ID NO: 21 - DNA, 22 bp, synthetic construct
  misc_feature 1..22: note = CHEMICALLY SYNTHESIZED
  source 1..22: mol_type = other DNA
acttgtaatgtaccacttcccg

SEQ ID NO: 22 - DNA, 20 bp, synthetic construct
  misc_feature 1..20: note = CHEMICALLY SYNTHESIZED
  source 1..20: mol_type = other DNA
cccctcaccacagagtctgc

SEQ ID NO: 23 - DNA, 24 bp, synthetic construct
  misc_feature 1..24: note = CHEMICALLY SYNTHESIZED
  source 1..24: mol_type = other DNA
aagggtgttgttgtcctcaatctt

SEQ ID NO: 24 - DNA, 28 bp, synthetic construct
  misc_feature 1..28: note = CHEMICALLY SYNTHESIZED
  source 1..28: mol_type = other DNA
ctcaaataaagttgaaatccttacaaac

SEQ ID NO: 25 - DNA, 22 bp, synthetic construct
  misc_feature 1..22: note = CHEMICALLY SYNTHESIZED
  source 1..22: mol_type = other DNA
tcttggtagggatgaattggac

SEQ ID NO: 26 - DNA, 21 bp, synthetic construct
  misc_feature 1..21: note = CHEMICALLY SYNTHESIZED
  source 1..21: mol_type = other DNA
gggaatctgagtggacatgtg

SEQ ID NO: 27 - DNA, 21 bp, synthetic construct
  misc_feature 1..21: note = CHEMICALLY SYNTHESIZED
  source 1..21: mol_type = other DNA
ccagaattgagtgcgttgatg

SEQ ID NO: 28 - DNA, 20 bp, synthetic construct
  misc_feature 1..20: note = CHEMICALLY SYNTHESIZED
  source 1..20: mol_type = other DNA
acagaaacgcttttgcaagg

SEQ ID NO: 29 - DNA, 20 bp, synthetic construct
  misc_feature 1..20: note = CHEMICALLY SYNTHESIZED
  source 1..20: mol_type = other DNA
ggtgagccatcttttgcatg

SEQ ID NO: 30 - DNA, 21 bp, synthetic construct
  misc_feature 1..21: note = CHEMICALLY SYNTHESIZED
  source 1..21: mol_type = other DNA
gcgagaaaatcacccactttg

SEQ ID NO: 31 - DNA, 20 bp, synthetic construct
  misc_feature 1..20: note = CHEMICALLY SYNTHESIZED
  source 1..20: mol_type = other DNA
tggctggaaataaccgtgag

SEQ ID NO: 32 - DNA, 19 bp, synthetic construct
  misc_feature 1..19: note = CHEMICALLY SYNTHESIZED
  source 1..19: mol_type = other DNA
tgaattactgtcgctgggc

SEQ ID NO: 33 - DNA, 23 bp, synthetic construct
  misc_feature 1..23: note = CHEMICALLY SYNTHESIZED
  source 1..23: mol_type = other DNA
gtacaaccattttcaccgaacag

SEQ ID NO: 34 - DNA, 21 bp, synthetic construct
  misc_feature 1..21: note = CHEMICALLY SYNTHESIZED
  source 1..21: mol_type = other DNA
atcaatccgttctactcagcg

SEQ ID NO: 35 - DNA, 21 bp, synthetic construct
  misc_feature 1..21: note = CHEMICALLY SYNTHESIZED
  source 1..21: mol_type = other DNA
gacatacgccgatattccctg

SEQ ID NO: 36 - DNA, 22 bp, synthetic construct
  misc_feature 1..22: note = CHEMICALLY SYNTHESIZED
  source 1..22: mol_type = other DNA
ggaggtagcatcttgtacgaag

SEQ ID NO: 37 - DNA, 21 bp, synthetic construct
  misc_feature 1..21: note = CHEMICALLY SYNTHESIZED
  source 1..21: mol_type = other DNA
cggtatgcaggatctcaagtc

SEQ ID NO: 38 - DNA, 20 bp, synthetic construct
  misc_feature 1..20: note = CHEMICALLY SYNTHESIZED
  source 1..20: mol_type = other DNA
cgagtgtgattacagccagg

SEQ ID NO: 39 - DNA, 19 bp, synthetic construct
  misc_feature 1..19: note = CHEMICALLY SYNTHESIZED
  source 1..19: mol_type = other DNA
tgacaacgaacatccagcg

SEQ ID NO: 40 - DNA, 21 bp, synthetic construct
  misc_feature 1..21: note = CHEMICALLY SYNTHESIZED
  source 1..21: mol_type = other DNA
ctgtgttcactgcggattttg

SEQ ID NO: 41 - DNA, 21 bp, synthetic construct
  misc_feature 1..21: note = CHEMICALLY SYNTHESIZED
  source 1..21: mol_type = other DNA
ctcctgtgttttaagcgtgac

SEQ ID NO: 42 - DNA, 1828 bp, synthetic construct
  misc_feature 1..1828: note = CHEMICALLY SYNTHESIZED
  source 1..1828: mol_type = other DNA
atggcttcctctatgctctcttccgctactatggttgcctctccggctcaggccactatggtcgctcctttcaacggacttaagtcctccgctgccttcccagccacccgcaaggctaacggaggtggatcgctgggaatgtattctgaatcggcagtaaggaagaaaagtagccgaggctacgatgttccagagggagtggacattcggggacgttatgatgaagaatttgccaggattctcaacaaggaagccttgctgtttgtggctgatttacagaggactttcagaaaccacataaggtattcgatggaatgccgcagagaagccaaaaggaggtacaatgaaggggcggtgccggggtttgatccggcgaccaagtatataagggaatctgagtggacatgtgcatcagtccccccggcagttgctgatcggagagtggagatcaccggacctgtggagcggaagatgatcatcaacgcactcaattctggagctaaagttttcatggcggactttgaagatgcactatcaccaaactgggagaatttgatgagggggcaaattaatctgaaggatgcagttgatgggactataagcttccatgacaaagctagaaacaaggtttataaactgaacgatcagacagccaagctctttgttcgccctcgaggttggcacttcgctgaggctcatatcttcatcgacggcgagcctgccaccggctgtcttgtggacttcgggctctacttttttcacaaccatgccaatttccggcgctctcaaggtcaaggttctggccctttcttttaccttcccaaaatggagcactccagggaagcaaaaatatggaacagtgtatttgagagagcagagaagatggcagggatagagaggggcagcatcagggccactgtgctgattgaaacacttccagcagtgtttcaaatggatgaaatactctatgagctgagggatcattctgtgggattgaactgtggtagatgggattacatattcagctatgtcaagaccttccaggctcacctagatcgcctgttacccgaccgagtccaagtcggtatggcacaacatttcatgaggagttattctgatctccttatcaggacttgtcatacggttgtgtgccacgtgggaggcatggctgctcaaattccaattagggatgacccgaaggcaaatgagatggcacttgagctagtgaggaaggacaaattgagagaggcaaaggcaggacatgatggaacatgggcagcacatccaggattaatcccagcatgtatggaagtgtttaccaacagcatgggaaatgcccccaatcagatccgatctgcaagacgcgatgatgctgcaaacctaactgaggatgacctcttgcagcaaccgaggggtgttcgtacattggaagggctccggttgaacacccgagtcggaattcagtacctagcagcatggctaaccgggacaggctctgtgcctctctacaaccttatggaagatgcagccacagctgaaatcagcagggttcaaaactggcaatggctgaagtatggagtggaattggatggagatgggcttggagtgagagtgaacaaggaactgttcgcaagagtggtggaagaagaaatggaaaggattgaaagagaagtggggaaggagaaattcaggaagggaatgtacaaagaggcttgcaagatgttcacaaggcaatgcacagcgccaaccttggatgattttctgaccttggatgcgtacaaccacatagtcatacatcatcccagggagctgtccaggctctgagctt

SEQ ID NO: 43 - protein, 607 aa, synthetic construct
  REGION 1..607: note = CHEMICALLY SYNTHESIZED
MASSMLSSATMVASPAQATMVAPFNGLKSSAAFPATRKANGGGSLGMYSESAVRKKSSRGYDVPEGVDIRGRYDEEFARILNKEALLFVADLQRTFRNHIRYSMECRREAKRRYNEGAVPGFDPATKYIRESEWTCASVPPAVADRRVEITGPVERKMIINALNSGAKVFMADFEDALSPNWENLMRGQINLKDAVDGTISFHDKARNKVYKLNDQTAKLFVRPRGWHFAEAHIFIDGEPATGCLVDFGLYFFHNHANFRRSQGQGSGPFFYLPKMEHSREAKIWNSVFERAEKMAGIERGSIRATVLIETLPAVFQMDEILYELRDHSVGLNCGRWDYIFSYVKTFQAHLDRLLPDRVQVGMAQHFMRSYSDLLIRTCHTVVCHVGGMAAQIPIRDDPKANEMALELVRKDKLREAKAGHDGTWAAHPGLIPACMEVFTNSMGNAPNQIRSARRDDAANLTEDDLLQQPRGVRTLEGLRLNTRVGIQYLAAWLTGTGSVPLYNLMEDAATAEISRVQNWQWLKYGVELDGDGLGVRVNKELFARVVEEEMERIEREVGKEKFRKGMYKEACKMFTRQCTAPTLDDFLTLDAYNHIVIHHPRELSRL

SEQ ID NO: 44 - DNA, 3410 bp, synthetic construct
  misc_feature 1..3410: note = CHEMICALLY SYNTHESIZED
  source 1..3410: mol_type = other DNA
aatggcttcctctatgctctcttccgctactatggttgcctctccggctcaggccactatggtcgctcctttcaacggacttaagtcctccgctgccttcccagccacccgcaaggctaacggaggtccacgcggccagggcaagcgcctggctcagctccttggagctcagctgaagcagtacgcagcggaggtgcgtggcatcagcacagctggtggcgcttctcgcggtggagctcgaggacctgcatcccctagctcgctagagcagcagacgcgccaggtcgctcaggttgctgttcagcagtcgactcagcaggcagtgaaggtcgttgtgccggccatcaaagtagacctggttggtgcggtcagctcggtgtctgagagcgacaaggtggagccgggtgtgttcaagaacgtggatggccaccgcttcgaggacggtcgctatgccgcttttgttgaggagattacaaagtttatccccaaggagcgccagtactcggaccccgtgcgcacattcgcgtatggcacggatgcctccttctaccggcttaacccgaagctggtagtgaaggtgcacaacgaggacgaggtccgccgcatcatgcccatcgcggagcggctgcaggtccctatcaccttccgcgcggccggcacgtcgctgtctgggcaggcaattaccgactcggtgctcattaagctgagccacacgggcaagaacttccgcaactttaccgtgcacggcgacggtagcgtgatcacggtggagccgggcctcattggcggcgaggtgaaccgcatcctggcggcacaccagaagaagaacaagctgcccatccagtacaagatcggacccgacccctcctccatcgacagctgcatgatcggcggcatcgtgtccaacaacagcagcggcatgtgctgcggcgtgagccagaacacctaccacacgctgaaggacatgcgggtggtgttcgtagacggaacggtgctggacacggccgaccccaactcgtgcaccgccttcatgaagagccaccgctcgctggtggatggcgtcgtgagcctggcgcgccgcgtgcaggccgacaaggagctgacggcgctcatccgccgcaagttcgccatcaagtgcaccaccggctactccctgaacgcgctggtggacttcccggtggacaaccccattgagatcatcaagcacctcatcatcggcagcgagggcacgctgggcttcgtcagccgcgccacctacaacaccgtgcccgagtggcccaacaaggcctcggccttcatcgtgttcccggacgtgcgcgccgcctgcaccggcgcctcggtgctgcgcaacgaaacctccgtggacgcggtggagctgtttgaccgcgccagcctgcgcgagtgcgagaacaacgaggacatgatgcgcctggtgcccgacatcaagggctgcgaccccatggcggcagcgctgctgatcgagtgccgcggccaggacgaggccgcactgcagagccgcattgaggaggtggtgcgcgtgctgacggcggcgggcctgcccttcggcgccaaggccgcgcagcccatggccatcgacgcctaccccttccaccacgaccagaagaacgccaaggtcttttgggacgtgcgcaggggcctgatccccattgtgggcgcggcgcgcgagcccggcacatccatgctgatcgaggacgtggcctgccccgtggacaagctggccgacatgatgatcgacctgatcgacatgttccagcgccacggctaccacgacgcctcctgcttcggccacgcgctcgagggcaaccttcatttggtattctcgcagggcttccgcaacaaggaggaggtgcagcgcttcagcgacatgatggaggagatgtgccatctggtagccaccaagcactcgggcagcctcaagggcgagcacggcacgggccgcaacgtggcgccgttcgtggagatggagtggggcaacaaggcgtacgagctgatgtgggagctcaaggcgctgttcgaccccagccacaccctcaacccgggcgtcatcctcaaccgcgaccaggacgcgcacatcaagttcctgaagccctcgcccgcggcctcgcccatcgtcaaccgctgcatcgagtgcggcttctgcgagtccaactgcccctcgcgcgacatcacgctcacgccgcgccagcgcatctccgtgtaccgcgagatgtaccgcctcaagcagctgggcccgggcgccagcgaggaggagaagaagcagctggcggccatgagcagctcgtacgcctacgacggcgagcagacgtgcgcggcggacggcatgtgccaggagaagtgccccgtcaagatcaacacgggcgatctgatcaagtcgatgcgtgccgagcacatgaaggaggagaaaaccgccagcggcatggcagactggctggccgccaacttcggcgtcatcaactccaacgtgccgcgcttcctcaacatcgtcaacgccatgcatagcgtagtgggctcggcgcctctgtccgccatcagccgcgcgctcaacgccgccaccaaccatttcgtaccggtgtggaacccctacatgcccaagggcgcggcgccgctcaaggtgcccgccccgccggcgccggcagctgctgaggcctcgggcatcccgcgcaaggtggtgtacatgcccagctgcgtgacgcgcatgatgggccccgccgcctccgacaccgaaaccgcggcggtgcacgagaaggtgatgagcctgttcggcaaggccggctacgaggtgatcatccccgagggcgtggccagccagtgctgcggcatgatgttcaacagccgcggcttcaaggacgccgccgccagcaagggcgcggagctggaggcggcgctgctcaaggcctcggacaatggcaagatccccatcgtcatcgacacctcgccctgcctggcgcaggtgaagagccagatcagcgagccgtcgctgcgcttcgcgctgtacgagccggttgagttcatccggcacttcctggtggacaagctggagtggaagaaggtgcgcgaccaggtggccatccacgtgccctgctcctccaagaagatgggcatcgaggagtccttcgcgaagctggcgggcctgtgcgccaacgaggtggtgccctcgggcattccttgctgcggcatggcgggcgaccgcggcatgcgcttccccgagctgaccggcgcctcgctgcagcacctcaacctgcccaagacctgcaaggacggctactccaccagccgcacctgcgagatgtcgctcagcaaccacgccggcatcaacttcaggggcctggtgtacctggtggatgaggccacggcgcctaagaagcaggccgccgctgccaagaccgcgt

SEQ ID NO: 45 - protein, 1136 aa, synthetic construct
  REGION 1..1136: note = CHEMICALLY SYNTHESIZED
MASSMLSSATMVASPAQATMVAPFNGLKSSAAFPATRKANGGPRGQGKRLAQLLGAQLKQYAAEVRGISTAGGASRGGARGPASPSSLEQQTRQVAQVAVQQSTQQAVKVVVPAIKVDLVGAVSSVSESDKVEPGVFKNVDGHRFEDGRYAAFVEEITKFIPKERQYSDPVRTFAYGTDASFYRLNPKLVVKVHNEDEVRRIMPIAERLQVPITFRAAGTSLSGQAITDSVLIKLSHTGKNFRNFTVHGDGSVITVEPGLIGGEVNRILAAHQKKNKLPIQYKIGPDPSSIDSCMIGGIVSNNSSGMCCGVSQNTYHTLKDMRVVFVDGTVLDTADPNSCTAFMKSHRSLVDGVVSLARRVQADKELTALIRRKFAIKCTTGYSLNALVDFPVDNPIEIIKHLIIGSEGTLGFVSRATYNTVPEWPNKASAFIVFPDVRAACTGASVLRNETSVDAVELFDRASLRECENNEDMMRLVPDIKGCDPMAAALLIECRGQDEAALQSRIEEVVRVLTAAGLPFGAKAAQPMAIDAYPFHHDQKNAKVFWDVRRGLIPIVGAAREPGTSMLIEDVACPVDKLADMMIDLIDMFQRHGYHDASCFGHALEGNLHLVFSQGFRNKEEVQRFSDMMEEMCHLVATKHSGSLKGEHGTGRNVAPFVEMEWGNKAYELMWELKALFDPSHTLNPGVILNRDQDAHIKFLKPSPAASPIVNRCIECGFCESNCPSRDITLTPRQRISVYREMYRLKQLGPGASEEEKKQLAAMSSSYAYDGEQTCAADGMCQEKCPVKINTGDLIKSMRAEHMKEEKTASGMADWLAANFGVINSNVPRFLNIVNAMHSVVGSAPLSAISRALNAATNHFVPVWNPYMPKGAAPLKVPAPPAPAAAEASGIPRKVVYMPSCVTRMMGPAASDTETAAVHEKVMSLFGKAGYEVIIPEGVASQCCGMMFNSRGFKDAAASKGAELEAALLKASDNGKIPIVIDTSPCLAQVKSQISEPSLRFALYEPVEFIRHFLVDKLEWKKVRDQVAIHVPCSSKKMGIEESFAKLAGLCANEVVPSGIPCCGMAGDRGMRFPELTGASLQHLNLPKTCKDGYSTSRTCEMSLSNHAGINFRGLVYLVDEATAPKKQAAAAKTA

SEQ ID NO: 46 - RNA, 694 nt, synthetic construct
  misc_feature 1..694: note = CHEMICALLY SYNTHESIZED
  source 1..694: mol_type = other RNA
tactattccagcagtaagacagagcctactggcaatcgtcggctgagctggcagtcgcatccatctttcttcataccaattcgatcattctgttggaagtgccagcaaaatcacccgagttgatgttcagaaggcaatcaatgacgtgctgtttcttgacagagaagatgcatgatggtgtttataaagttaagttttcactgtttctcgatcaactcaatgattaggaactctcctgtctatctagttttgggtttacataggatctatatagaggtaaatgtccatcccacctttgtattttctggaagaaagaaagataagacactgagaataaccgcgagatagagaggcttaaagatggagaacaaagaagggtttttgaatattacaaaatacaaaggtgggatggacatttacctctatatagatcctatgtaaacccaaaactagatagacaggagagttcctaatcattgagttgatcgagaaacagtgaaaacttaactttataaacaccatcatgcatcttctctgtcaagaaacagcacgtcattgattgccttctgaacatcaactcgggtgattttgctggcacttccaacagaatgatcgaattggtatgaagaaagatggatgcgactgccagctcagccgacgattgccagtaggctctgtcttactgctggaatgc

(19) United States
(12) Patent Application Publication
(10) Pub. No.: US 2026/0078393 A1
(43) Pub. Date: Mar. 19, 2026

(54) PLANTS WITH INCREASED PHOTORESPIRATION EFFICIENCY

(71) Applicant: The United States of America, as Represented by the Secretary of Agriculture, Washington, DC (US)

(72) Inventors: Donald R. ORT, Champaign, IL (US); Paul F. SOUTH, Champaign, IL (US); Berkley WALKER, Dusseldorf (DE)

(73) Assignee: The United States of America, as Represented by the Secretary of Agriculture, Washington, DC (US)

(21) Appl. No.: 19/323,550

(22) Filed: Sep. 9, 2025

Related U.S. Application Data
(63) Continuation of application No. 15/913,395, filed on Mar. 6, 2018, now Pat. No. 12,534,736.
(60) Provisional application No. 62/467,993, filed on Mar. 7, 2017.

(51) Int. Cl. C12N 15/82

(52) U.S. Cl. CPC C12N 15/8218; C12N 15/8262; C12N 15/8269

(57) ABSTRACT

Presented herein are plants with altered photorespiratory characteristics. Disruption of transport proteins involved in shuttling glycolate and/or glycerate results in reductions in photosynthetic rates, reduced plant growth and alterations in gene expression and photosynthetic metabolite profiles. Such disruptions are also combined with introduced genes expressing components of alternate photorespiratory enzyme pathways to increase photosynthetic efficiency.

### CROSS-REFERENCE TO RELATED APPLICATIONS

This application is a continuation of U.S. application Ser. No. 15/913,395, filed on Mar. 6, 2018, which claims the benefit of priority under 35 U.S.C. § 119(e) to U.S. Provisional Application No. 62/467,993, filed on Mar. 7, 2017, the contents of each of which are hereby incorporated by reference in their entirety.

### REFERENCE TO AN ELECTRONIC SEQUENCE LISTING

The contents of the electronic sequence listing (794542001501SEQLIST.xml; Size: 73,607 bytes; and Date of Creation: Sep. 3, 2025) is herein incorporated by reference in its entirety.

### FIELD

The present disclosure provides plants with altered photorespiratory characteristics. Disruption of transport proteins involved in shuttling glycolate and/or glycerate results in reductions in photosynthetic rates, reduced plant growth and alterations in gene expression and photosynthetic metabolite profiles. Such disruptions, when combined with introduced genes expressing components of alternate photorespiratory enzyme pathways, increase photosynthetic efficiency.

### BACKGROUND

Ribulose-1, 5-bisphosphate carboxylase/oxygenase (RubisCO) catalyzes the fixation of ribulose-1, 5-bisphosphate (RuBP) with CO₂ generating two molecules of 3-phosphoglycerate (3-PGA). However, at 25° C. and current CO₂ levels about 25% of RubisCO catalytic activity in plants with C3 photosynthetic metabolism is the fixation of the competing substrate oxygen instead of carbon dioxide, resulting in the conversion of RuBP to one molecule of 3-PGA and one molecule of 2-phosphoglycolate (2-PG) (Bowes et al., Biochem Bioph. Res. Co (1971) 45:716-22; Ogren and Bowes, Nature-New Biol. (1971) 230:159-60; Lorimer, G. H., Ann. Rev. Plant Physiol. Plant Mol. Biol. (1981) 32:349-83; Ogren, W. L., Ann. Rev. Plant Physiol. Plant Mol. Biol. (1984) 35:415-42; Sharkey, T. D., Physiologia Plantarum (1988) 73:147-52). 2-PG accumulation in the chloroplast stroma can inhibit triose phosphate isomerase and phosphofructokinase thereby decreasing RuBP regeneration capacity (Anderson, L. E., Biochim Biophys Acta (1971) 235:237-44; Kelly and Latzko, Febs Lett (1976) 68:55-58). Although 2-PG is rapidly dephosphorylated by 2-phosphoglycolate phosphatase, the glycolate produced can also inhibit the rate of photosynthesis in the chloroplast and is considered toxic to the cell (Kelly and Latzko, supra; GonzalezMoro et al., J. Plant Physiol. (1997) 150:388-94). The inhibition of photosynthesis by 2-PG/glycolate is prevented and partial recovery of the reduced carbon is accomplished through the C2 photorespiratory pathway involving steps in the chloroplast, peroxisome, mitochondria and the cytosol (Somerville and Ogren, Plant Physiol (1979) 63:152; Eisenhut et al., Plant Biol. (2013) 676-85). Photorespiration converts two molecules of 2-PG to one molecule of 3-PGA and releases one molecule of CO₂.

In addition, the photorespiratory cycle utilizes ATP and, as a byproduct of the conversion of glycine to serine, produces ammonia (NH₃) in the mitochondria. Plants then recycle the NH₃ using reducing equivalents NAD(P)H. As a result, photorespiration under current atmospheric CO₂ concentrations results in a ˜15 to 50% drag on seasonal C3 photosynthetic efficiency depending upon regional growing season temperature (Ogren, supra; Peterhansel et al., Photorespiration. The Arabidopsis Book (2010), 20130). Losses in yield due to photorespiration add up to ˜150 trillion calories per year in midwestern US soybean and wheat production alone (Walker et al., Ann. Rev. Plant Biol. (2016) 107-29), and has similar negative impacts on other major C3 crops such as rice and potato (Sharkey, T. D., supra, Zhu et al., Ann. Rev. Plant Biol. (2010) 61:235-61).

Photorespiration is essential for C3 plants but operates at the massive expense of fixed carbon dioxide and energy. Photorespiration is initiated when the initial enzyme of photosynthesis, ribulose-1,5-bisphosphate carboxylase/oxygenase (Rubisco), reacts with oxygen instead of carbon dioxide and produces the toxic compound glycolate that is then recycled by photorespiration. Photorespiration can be modeled at the canopy and regional scales to determine its cost under current and future atmospheres. A regional-scale model reveals that photorespiration currently decreases US soybean and wheat yields by 36% and 20%. Even modest improvements in this photorespiratory loss could be worth $100s million annually in the US alone making photorespiration a target process for improving crop yield (Annu. Rev. Plant Biol. 2016. 67:107-29). Advances in synthetic biology have enabled the introduction of several novel pathways into plant chloroplasts intending to short circuit the native pathway by introducing enzymes that metabolize glycolate in the chloroplast using less energy and shifting the location of photorespiratory CO₂ production from the mitochondria to the chloroplast thereby enabling rapid refixation by Rubisco (Kebeish et al., Nat Biotechnol (2007) 25:593-599; Maurino and Peterhansel, Curr Opin Plant Biol (2010) 13: 249-256).

The soluble enzymes involved in photorespiration have been well studied over the past four decades providing much information on the biochemistry and genetics governing photorespiratory metabolism (Peterhansel et al., supra; Timm and Bauwe, Plant Biol. (2013) 15:737-47). In contrast, only a small number of transporters have been demonstrated to be involved in photorespiration despite at least 25 proposed transport steps involved in the recycling of carbon in photorespiration (Eisenhut et al., supra). Importantly, photorespiration is a high flux pathway that interacts with multiple other metabolic pathways including the nitrogen cycle and amino acid biosynthesis (Fernie et al., Plant Biol. (2013) 15:748-53).

The first transporters identified to be involved in photorespiration were the chloroplastic dicarboxylate transporters DiT1 and DiT2 (Woo et al., Plant Physiol. (1987) 84:624-32). A single point mutation in DiT2.1 and subsequent biochemical characterization revealed that DiT2 is the glutamate/malate transporter located in the chloroplast inner envelope membrane (Renne et al., Plant J. (2003) 35:316-31). Antisense repression of DiT1 demonstrated the classical photorespiratory mutant phenotype of decreased growth under ambient CO₂ and complementation by elevated carbon dioxide concentration ([CO₂]) and resulted in reduced nitrate re-assimilation due to a decrease in 2-oxoglutarate transport in the chloroplast (Schneidereit et al., Plant J. (2006) 45:206-24). Together, DiT1 and DiT2 are necessary for the proper refixation of released ammonia from the glycine decarboxylation reaction in photorespiratory metabolism.

More recently, co-expression analysis was used to identify other potential transporters involved in photorespiration (Bordych et al., Plant Biol. (2013) 15:686-93). Co-expression analysis identified A BOUT DE SOUFFLE (BOU) a mitochondrial transporter required for normal glycine decarboxylase (GDC) activity and meristematic growth in which null mutants exhibit the photorespiratory mutant phenotype of complementation by elevated [CO₂](Eisenhut et al., Plant J. (2013) 73:836-49). Currently, the only photorespiratory pathway transporter that transports carbon derived directly from glycolate that has been identified is the plastidic glycolate/glycerate translocator protein, PLGG1. Plgg1 is co-expressed with many enzymes involved in photorespiration (Pick et al., Proc. Nat'l Acad. Sci. USA (2013) 110:3185-90). A Plgg1 T-DNA knockout line in Arabidopsis thaliana (plgg1-1) reveals the role of PLGG1 in the first and final transport steps in the photorespiratory pathway, viz, the export of glycolate from and import glycerate into the chloroplast (Pick et al., supra). Nearly 30 years prior to the molecular identification of PLGG1, the export of glycolate coincident with the import of glycerate import had been demonstrated in purified spinach chloroplasts (Howitz and McCarty, Biochem. (1985) 24:3645-50; Howitz and McCarty, Plant Physiol. (1986) 80:390-95; Howitz and McCarty, Plant Physiol. (1991) 96:1060-69; Young and McCarty, Plant Physiol. (1993) 101:793-99). Additionally, PLGG1 was identified as a chloroplast protein in proteomic studies and was originally thought to be involved in programmed cell death, though current evidence suggests the phenotype was linked to accumulation of photorespiratory intermediates (Yang et al., New Phytol. (2012) 193:81-95; Pick et al., supra). However, it was shown that the Arabidopsis plgg1-1 line showed no differences in the quantum efficiency of CO₂ assimilation, or changes in the photorespiratory CO₂ compensation point compared to wild type when measured under low light conditions (Walker et al., Photosyn Res. (2016) 129:93-103). Combined, these data show that PLGG1 protein is involved in photorespiratory metabolism but also suggest an additional pathway for glycolate to exit the chloroplast, as well as demonstrate the difficulty in phenotypically identifying transporters in the photorespiration pathway (Hodges et al., J. Exp. Bot. (2016) 3015-26).

Although both genetic and co-expression approaches have been successful in identifying genes involved in photorespiratory metabolism, many of the transporters involved in the flux of photorespiratory intermediates remain unknown. An alternative approach to co-expression analysis is to identify candidate chloroplast inner membrane transporters from chloroplast envelope proteomic studies and screen tDNA insertional mutants of the candidate genes for a photorespiratory mutant phenotype using chlorophyll fluorescence (Badger et al., Funct. Plant Biol. (2009) 36:867-73; Sun et al., Nucleic Acids Res., (2009) 37:D969-D974). Photorespiration deficient mutants exhibit a reduction in Fv/Fm chlorophyll fluorescence due to impaired function of photosystem II (PSII) when exposed to illumination under low CO₂ levels (Kozaki and Takeba, Nature (1996) 384:557-60; Wingler et al., Philosoph. Trans. Royal Soc. B-Biol. Sci. (2000) 355:1517-29; Takahashi et al., Plant Physiol. (2007) 144:487-94).

Using this high throughput fluorescence-based approach in combination with forward genetics targeting putative transporter-like chloroplast inner envelope membrane proteins has the potential to identify additional genes important for photorespiratory metabolite transport. Bile acid sodium symporters are a family of transport proteins which were first identified as bile acid transporters in the mammalian liver. Further analysis showed that the BASS family of transporters exhibit a broad range of substrate specificity including non-bile acid organic compounds such as pyruvate, steroids, and xenobiotics (Furumoto et al., Nature (2011) 476:472-75; Claro da Silva et al., Mol. Aspects of Med. (2013) 34:252-69). Although bile acids are not produced in plants, BASS family genes are present in both monocots and dicots (Gigolashvili et al., The Plant Cell (2009) 21:1813-29; Sawada et al., Plant and Cell Physiol. (2009) 50:1579-86; Furumoto et al., supra).

As detailed herein, the Bile Acid Sodium Symporter 6 protein (BASS6) as a glycolate transporter involved in photorespiration has been identified. Analysis of bass6 knockout T-DNA lines in Arabidopsis (bass6-1 and bass6-2) revealed that loss of Bass6 resulted in a photorespiratory mutant phenotype and accumulation of photorespiratory metabolic intermediates glycine and glycolate. In addition, BASS6 protein localized to the chloroplast envelope and the capacity of BASS6 to transport glycolate was demonstrated through combined yeast complementation and transport analysis. A bass6-plgg1 double mutant showed additive growth defects.

Our discovery has revealed that photorespiratory short circuits or bypass pathways have been only modestly effective due to the rapid export of glycolate out of the chloroplast via the two glycolate transporters located in the chloroplast envelope membrane. PLGG1 (Proc Natl Acad Sci USA (2013) 110(8):3185-90) is a plastidal glycolate glycerate translocator that exchanges glycolate for glycerate across the chloroplast envelope membrane. While PLGG1 is wholly responsible for glycerate import, BASS6 and PLGG1 share glycolate export from the chloroplast. Thus the combined activity of BASS6 and PLGG1 compete with the synthetic photorespiratory bypass pathway for glycolate thereby limiting the effectiveness of the bypass in improving photosynthetic efficiency and plant growth/yield.

Toxic byproducts of RuBisCO oxygenation reaction and glycine conversion in photorespiration (glycolate and ammonia respectively) are re-fixed and converted into usable products at a high-energy demand and a net loss of fixed carbon, among three organelles: the chloroplast, the peroxisome, and the mitochondria (Bauwe et al, Trends Plant Sci. (2010) 15:330-6). Some photosynthetic algae, bacteria, and plants have evolved ways to reduce the stress of photorespiration via carbon concentrating mechanisms (CCM) and C4 photosynthesis (Price et al, J. Exp. Bot. (2013) 64:753-68). As an alternative, bypassing photorespiration using alternative metabolic pathways could reduce the energy demand and re-capture the carbon lost in the process more efficiently (Betti et al., J. Exp. Bot. (2016) 67:2977-88). Three different photorespiration bypasses have been demonstrated in plants such as Arabidopsis, Camelina sativa, and potato (Dalal et al, Biotechnol. Biofuels (2015) 8; Kebeish et al, Nat. Biotechnol. (2007) 593-9; Maier et al, Front. Plant Sci. (2012) 3:12; Nolke et al, Plant Biotechnol. J. (2014) 12:734-42). Although these bypasses, including some modifications, showed improvements in plant productivity, there has been no demonstration of their effectiveness under agricultural condition and no current attempt to fully optimize a bypass to photorespiration for a farmer's field.

To address these concerns, presented herein are plants, and methods for producing them, that lack chloroplast glycolate export capability as well as those containing one or more alternate photorespiratory bypass pathway(s) to increase photosynthetic efficiency. A combination of the two approaches results in additional efficiency.

### SUMMARY

Provided herein are genetically altered plants, containing one or more genetic alterations resulting in the loss or reduction of the ability of the plant to transport glycolate from at least a portion of the chloroplasts and resulting in the gain of the ability to convert glycolate to energy within at least a portion of the chloroplasts of the plant. In one embodiment, the loss of chloroplast glycolate transport ability results from lack of production of a functional protein with at least 70% identity to SEQ ID NO:6. In other embodiments, the loss of chloroplast glycolate transport ability comprises inducing RNA interference by the expression of an RNA molecule at least 95% identical to SEQ ID NO: 46. In still other embodiments, the gain of the ability to convert glycolate to energy within the chloroplasts comprises the production of a transgenic malate synthase and a transgenic glycolate dehydrogenase in the chloroplasts. In specific embodiments, the malate synthase is at least 95% identical to amino acid residues 41-607 of SEQ ID NO: 43 and the glycolate dehydrogenase is at least 95% identical to amino acid residues of 41-1136 of SEQ ID NO: 45. In a particular embodiment, the malate synthase comprises SEQ ID NO: 43 and the glycolate dehydrogenase comprises SEQ ID NO: 45. In a another specific embodiment, the loss of chloroplast glycolate transport ability comprises lack of production of a protein with at least 95% identity to SEQ ID NO:3 and lack of production of a protein with at least 95% identity to SEQ ID NO:6; and wherein the gain of the ability to convert glycolate to energy within the chloroplasts comprises the production of a protein with at least 95% identity to SEQ ID NO:43 and the production of a protein with at least 95% identity to SEQ ID NO:45. The genetically altered plant can be any C3 plant. For example, in some embodiments, plants of the present disclosure are rice, soybean, potato, cowpea, barley, wheat, or cassava.

Disclosed herein is also a method of producing a plant with increased growth or productivity, by: a) introducing a genetic alteration to the plant comprising a loss of the ability to transport glycolate from at least a portion of the chloroplasts of the plant; and b) introducing a genetic alteration to the plant comprising a gain of the ability to convert glycolate to energy within the chloroplasts, thereby increasing growth or productivity of the plant. In some embodiments, the loss of the ability to transport glycolate from at least a portion of the chloroplasts of the plant comprises the lack of production of a functional protein with at least 95% identity to SEQ ID NO:3, the lack of production of a functional protein with at least 95% identity to SEQ ID NO:6, or both. In still additional embodiments, the gain of the ability to convert glycolate to energy within the chloroplasts comprises the production of a transgenic malate synthase and a transgenic glycolate dehydrogenase in the chloroplasts. In some embodiments, the malate synthase is at least 95% identical to amino acid residues 41-607 of SEQ ID NO: 43 and the glycolate dehydrogenase is at least 95% identical to amino acid residues of 41-1136 of SEQ ID NO: 45. In specific embodiments, the malate synthase comprises SEQ ID NO: 43 and the glycolate dehydrogenase comprises SEQ ID NO: 45. According to a particular embodiment, the loss of chloroplast glycolate transport ability comprises lack of production of a protein with at least 95% identity to SEQ ID NO:3; lack of production of a protein with at least 95% identity to SEQ ID NO:6, or both; and wherein the gain of the ability to convert glycolate to energy within the chloroplasts comprises the production of a protein with at least 95% identity to SEQ ID NO:43 and the production of a protein with at least 95% identity to SEQ ID NO:45. Any C3 plant may be used with the methods of the present disclosure. In some embodiments, the plant is rice, soybean, potato, cowpea, barley, wheat, or cassava.

An additional embodiment provided herein is a genetically altered plant, comprising a first heterologous polynucleotide encoding a malate synthase and a second heterologous polynucleotide encoding a glycolate dehydrogenase, wherein the malate synthase and the glycolate dehydrogenase localize to a chloroplast of the plant. In preferred embodiments, the plant converts glycolate to energy within the chloroplast of the plant. In some embodiments, the malate synthase is from any source provided herein, including Cucurbita maxima. In particular embodiments, the malate synthase is at least 95% identical to amino acid residues 41-607 of SEQ ID NO: 43. In further embodiments, any of these plants expresses a glycolate dehydrogenase from an organism selected from any source provided herein, including Chladymonas reinhardtii. In specific embodiments, the glycolate dehydrogenase is at least 95% identical to amino acid residues 41-1136 of SEQ ID NO: 45. In a particular embodiment, the first heterologous polynucleotide encodes the amino acid sequence of SEQ ID NO: 43 and the second heterologous polynucleotide encodes the amino acid sequence of SEQ ID NO: 45. In some embodiments, the plant further comprises a reduced level, a reduced activity, a partial loss of activity, or a complete loss of activity of one or more endogenous glycolate transport proteins in a chloroplast of the plant. In some embodiments, the plant has a reduction or loss of glycolate transport from a chloroplast of the plant. In specific embodiments, the one or more glycolate transport proteins include PLGG1 and BASS6. In further embodiments, the one or more glycolate transport proteins have at least 70% sequence identity, at least 75% sequence identity, at least 80% sequence identity, at least 85% sequence identity, at least 90% sequence identity or at least 95% sequence identity to SEQ ID NO:6. In still further embodiments, at least one of the one or more glycolate transport proteins had at least 70% sequence identity, at least 75% sequence identity, at least 80% sequence identity, at least 85% sequence identity, or at least 90% sequence identity to SEQ ID NO:3. In other embodiments, at least one of the one or more glycolate transport protein had at least 95% sequence identity to SEQ ID NO:3. In some embodiments, the plant comprises a mutation in a DNA molecule encoding the glycolate transport protein. In additional embodiments, the plant comprises a heterologous polynucleotide encoding an RNA molecule that inhibits expression of the glycolate transport protein, such as one that is at least 95% identical to SEQ ID NO: 46. In further embodiments the reduced level, the reduced activity, the partial loss of activity, or the complete loss of activity of at least one of the one or more glycolate transport proteins was generated using a technology selected from the group consisting of CRISPR/Cas, TALEN, Zn-finger nuclease, and RNAi.

Another aspect of the present disclosure is a genetically altered plant, wherein the plant comprises a first heterologous polynucleotide encoding a first polypeptide having at least 95% identity to SEQ ID NO:43 and a second heterologous polynucleotide encoding a second polypeptide having at least 95% identity to SEQ ID NO:45, wherein the first polypeptide and the second polypeptide localize to a chloroplast of the plant. In additional embodiments, the plant further comprises a reduced level of or a reduced activity of a third polypeptide having at least 95% identity to SEQ ID NO:3 and a reduced level of or a reduced activity of a fourth polypeptide having at least 95% identity to SEQ ID NO:6. Exemplary plants include rice, soybean, potato, cowpea, barley, wheat, and cassava.

An additional aspect of the present disclosure provides a method of producing a plant with increased growth or productivity, comprising introducing a first heterologous polynucleotide encoding a malate synthase and a second heterologous polynucleotide encoding a glycolate dehydrogenase to the plant, wherein the malate synthase and the glycolate dehydrogenase localize to a chloroplast of the plant, wherein the plant has an increased ability to convert glycolate to energy within the chloroplast, thereby increasing growth or productivity of the plant. In some embodiments, this method also has a step of introducing a genetic alteration to the plant, wherein the plant has a reduced ability to transport glycolate from at least a portion of the chloroplasts of the plant. In additional embodiments, the malate synthase is from an organism provided herein, including Cucurbita maxima. In particular embodiments, the malate synthase is at least 95% identical to amino acid residues 41-607 of SEQ ID NO: 43. In further embodiments, the glycolate dehydrogenase is from an organism provided herein, including Chladymonas reinhardtii. In particular embodiments, the glycolate dehydrogenase is at least 95% identical to amino acid residues 41-1136 of SEQ ID NO: 45. In a specific embodiment, the malate synthase comprises the amino acid sequence of SEQ ID NO: 43 and the glycolate dehydrogenase comprises the amino acid sequence of SEQ ID NO: 45. In some embodiments, the step of introducing a genetic alteration to the plant that causes a reduced level, a reduced activity, a partial loss of activity, or a complete loss of activity of one or more endogenous glycolate transport proteins in a chloroplast of the plant. In some embodiments of this methodology, the reduced level, the reduced activity, the partial loss of activity, or the complete loss of activity of at least one of the one or more endogenous glycolate transport proteins comprises introducing a mutation into an endogenous DNA molecule that encoded the endogenous glycolate transport protein. In additional embodiments, the reduced level, the reduced activity, the partial loss of activity, or the complete loss of activity of at least one of the one or more endogenous glycolate transport proteins comprises introducing a heterologous polynucleotide encoding an RNA molecule that inhibits expression of the endogenous glycolate transport protein, such as where the RNA molecule is at least 95% identical to SEQ ID NO: 46. In particular embodiments, at least one of the one or more endogenous glycolate transport proteins is PLGG1 or BASS6. In some embodiments, at least one of the one or more endogenous glycolate transport protein has at least 70% sequence identity, at least 75% sequence identity, at least 80% sequence identity, at least 85% sequence identity, or at least 90% sequence identity to SEQ ID NO:6. In specific embodiments, at least one of the one or more endogenous glycolate transport protein has at least 95% sequence identity to SEQ ID NO:6. In other embodiments, at least one of the one or more endogenous glycolate transport protein has at least 70% sequence identity, at least 75% sequence identity, at least 80% sequence identity, at least 85% sequence identity, or at least 90% sequence identity to SEQ ID NO:3. In specific embodiments, at least one of the one or more endogenous glycolate transport protein has at least 95% sequence identity to SEQ ID NO:3. In particular embodiments, the one or more endogenous glycolate transport protein is a first glycolate transport protein having at least 95% identity to SEQ ID NO:6 and a second glycolate transport protein having at least 95% identity to SEQ ID NO: 3. In particular embodiments, the plant is rice, soybean, potato, cowpea, barley, wheat, or cassava.

### INCORPORATION BY REFERENCE

All publications, patents and patent applications mentioned in this specification are herein incorporated by reference to the same extent as if each individual publication, patent or patent application was specifically and individually indicated to be incorporated by reference.

### BRIEF DESCRIPTION OF THE DRAWINGS

The patent application file contains at least one drawing executed in color. Copies of this patent or patent application publication with color drawing(s) will be provided by the Office upon request and payment of the necessary fee.

The novel features of the disclosure are set forth with particularity in the claims. Features and advantages of the present disclosure are referred to in the following detailed description, and the accompanying drawings of which:

FIG. 1 provides a depiction of the photorespiratory C2 cycle.

FIG. 2 provides photographs of representative bass6 and plgg1 mutants compared to wild-type A. thaliana grown at ambient CO₂ (8 weeks 400 ppm CO₂ at 8 h light/16 h dark cycle (22° C./18° C.) at 250 μmol·m⁻²·s⁻¹ light intensity in growth chambers)

FIG. 3 provides a photograph of representative bass6 and plgg1 mutants compared to wild-type A. thaliana showing changes in Fv/Fm after 24 hours of low CO₂ and constant illumination. Numbers represent the average of 12 plants from three replicate experiments.

FIG. 4A and FIG. 4B provide graphs showing relative growth rate of bass6-1 and plgg1-1 mutants compared to wild-type A. thaliana at different CO₂ concentrations. In FIG. 4A, error bars indicate standard deviation and asterisk (*) indicates significant difference between CO₂ treatments. Double asterisk (**) indicates significant change in growth rate between T-DNA lines and WT. Statistical difference based on Student's T-test p<0.05.

FIG. 5 provides graphs showing reduction in assimilation (A), internal CO₂ concentration (Cᵢ), and stomatal conductance (gₛ) in A. thaliana mutants lacking Bass6. Photosynthetic measurements recorded at 400 ppm CO₂ and saturating light (1000 μmol·m⁻²·s⁻¹) for the indicated strains, assimilation (A), internal CO₂ concentration (Cᵢ) and stomatal conductance (gₛ). Letters indicate statistical differences based on ANOVA analysis N=3.

FIG. 6A and FIG. 6B provide analysis of a bass6, plgg1 double mutant A. thaliana showing additive photorespiratory phenotypic effects. FIG. 6A provides photographs showing representative wild-type, bass6, plgg1, and double mutant bass6, plgg1 growth and Fv/Fm changes in chlorophyll fluorescence. The photographs represent indicated plants grown for 4 weeks at 2000 ppm CO₂ then shifted to ambient CO₂ for 5 days. Fv/Fm images show changes in chlorophyll fluorescence due to the formation of chlorotic lesions on leaves. Images are representative of 5 repeats. FIG. 6B provides a graph showing the extent of chlorotic lesion formation in these plants. Area in cm² of leaf lesion size based on pixel density measured using photo software (Adobe).

FIG. 7 provides confocal microscopy images of isolated Nicotiana benthamiana protoplasts showing localization of GFP-tagged PLGG1 (panels D-F) and GFP-tagged BASS6 (panels G-I) to the chloroplast envelope. These images are maximum projections of 4 successive planes, and show that both PLGG1 and BASS6 localize in the chloroplast envelope (arrowheads) where they form stromules (starred arrowheads). All protoplasts also express P19 and a mCherry-tagged ER marker, not shown. Scale bars: 10 μm. Panels J-L provide light sheet images of N. benthamiana leaf tissue transiently expressing Bass6-eGFP expressed from the 35s promoter. Arrows indicate GFP fluorescence not associated with chlorophyll auto-fluorescence. Starred arrows indicate GFP fluorescence associated with the chlorophyll envelope. Scale Bars: 50 μm. In all panels, GFP signal is shown in green, while chloroplast auto-fluorescence is shown in magenta.

FIG. 8A and FIG. 8B provide analysis of a bass6, plgg1 double mutant A. thaliana showing additive photorespiratory phenotypic effects. FIG. 8A is a graph showing relative growth rates of indicated Arabidopsis T-DNA lines and either 2000 ppm (dark) or 400 ppm CO₂ (light) grey bars. Error bars indicate standard deviation of at least 5 plants per 3 biological replicates. Asterisk (*) indicates significant difference between CO₂ treatments. Double asterisk (**) indicates significant change in growth rate between T-DNA lines and WT. Statistical difference based on Student's T-test p<0.05. FIG. 8B is a graph of photosynthetic measurements recorded at indicated CO₂ concentration and saturating light (1000 μmol·m-2·s-1) for the indicated strains. Letters represent significant difference from ANOVA analysis and Tukey's post-hoc test. Error bars indicate standard deviation.

FIG. 9 provides a graph demonstrating accumulation of various photorespiratory intermediates in A. thaliana wild-type, bass6, plgg1, and double mutant bass6, plgg1 plants grown at elevated CO₂ for 6 weeks. Black bars indicate 2000 ppm and grey bars indicate 150 ppm CO₂. X-axis numbers represent relative differences of the indicated photorespiratory metabolite based on an internal standard. Error bars indicate standard error of the mean. Letters indicate statistical differences based on ANOVA analysis; N=3.

FIG. 10 provides graphs demonstrating the role of BASS6 and PLGG1 in glycolate metabolism in A. thaliana wild-type, bass6, plgg1, and double mutant bass6, plgg1. Indicated plant lines were grown in elevated CO₂ (2000 ppm) for 4 weeks then shifted to ambient air (400 ppm CO₂) for 24 hours. At the end of an 8-hour light cycle, tissue was collected as time 0. Each time point after was sample collection during the dark period. X-axis numbers represent relative differences of the indicated photorespiratory metabolite based on an internal standard. Error bars indicate standard error of the mean. Asterisks indicate statistical differences based on ANOVA analysis comparing WT control to T-DNA lines; N=3.

FIG. 11 provides photographs of various yeast strains expressing A. thaliana PLGG1 or BASS6 and showing the ability of both proteins to transport glycolate.

FIG. 12 provides a graph showing the ability of various yeast strains expressing A. thaliana PLGG1 or BASS6 to take up radio-labeled glycolate. Error bars indicate standard deviation and letters indicate statistical differences based on ANOVA analysis N=3.

FIG. 13 provides graphs demonstrating some of the genetic regulatory mechanisms controlling expression of BASS6 and PLGG1. Expression of Bass6 and Plgg1 in leaf tissue was determined in the plgg1-1 and the bass6-1 mutants by qRT-PCR analysis. In the left panel, error bars indicate standard error of the mean from 3 biological replicates including 3 technical replicates each. Asterisk indicates significant change (p<0.05). Relative growth rate of indicated Arabidopsis transgenic lines are shown in the right panel. Error bars indicate standard deviation of at least 5 plants per 3 biological replicates. Asterisk (*) indicates significant difference between transgenic lines grown under ambient air conditions. Statistical difference based on Student's T-test.

FIG. 14 Synthetic biology approach to photorespiration bypass. Model of three photorespiration bypass designs. Bypass 1 (orange) converts glycolate to glycerate using five genes from the E. coli glycolate pathway 3 genes DEF glycolate dehydrogenase, glyoxylate carboligase, and tartonic semialdehyde reductase. Bypass 2 (red/purple) utilizes three genes, glycolate oxidase, malate synthase, and catalase to remove hydrogen peroxide generated by glycolate oxidase. Bypass 3 (blue/purple) uses 2 genes. Chlamydomonas reinhadrtii glycolate dehydrogenase and Cucurbita maxima malate synthase.

FIG. 15A-15B. FIG. 15A provides representative photos of 9 day old transgenic tobacco lines during fluorescence-based screening for improved photorespiration bypass by changes in Fv′/Fm′ after 24 hours of low CO₂ and constant illumination. FIG. 15B provides combined values of the three bypass construct designs with and without RNAi targeting the glycolate/glycerate transporter PLGG1. Error bars indicate SEM. * indicates statistical difference compared to WT based on one-way ANOVA P<0.05.

FIG. 16A-16B Gene expression and protein analysis of Bypass 3 lines. FIG. 16A. qRT-PCR analysis of the two transgenes in Bypass 3 and the target gene PLGG1 of the RNAi construct. FIG. 16B. Western blot analysis using custom antibodies raised against the indicated target genes. 3 μg load of protein per lane except for the RbcS control (1.5 μg). Arrows ( [symbol] ) indicate detected protein based on molecular weight. Error bars indicate SEM. * indicates statistical difference compared to WT based on one-way ANOVA P≤0.05.

FIG. 17A-17B Gene expression analysis of bypass 1 and 2. FIG. 17A qRT-PCR analysis of the indicated transgenes and the native PLGG1 targeted for RNAi of bypass 1. Glycolate dehydrogenase subunits D,E,F (GDH), Tartonic acid semi-aldehyde reductase (TSR), Glyoxylate carboligase (GCL), Plastidic glycolate/glycerate transporter (PLGG1). FIG. 17B qRT-PCR analysis of the indicated transgenes and the native PLGG1 targeted for RNAi of bypass 2. Glycolate oxidase (GO), Catalase (CAT), Malate synthase (MS). Error bars indicated SEM.

FIG. 18A-18B. Field trial stem height and biomass. FIG. 18A Stem height analysis based on measurements recorded 7 weeks post germination. Error bars indicated SD and * indicated significance based on one-way ANOVA N=8. FIG. 18B. Percent difference in combined stem, leaf, and total dry weight biomass compared to WT control with and without the PLGG1 RNAi module. Error bars indicate SEM, * indicated significance based on one-way ANOVA N=8.

FIG. 19A-19C. Field trial photosynthetic efficiency. FIG. 19A. Combined apparent quantum efficiency of photosynthesis ((a) of bypass 1 determined by linear regression of assimilation based on available light response curves and saturating rates of assimilation of CO₂ at the indicated [CO₂]. FIG. 19B. Combined apparent quantum efficiency of photosynthesis (Φa) of bypass 2 determined by linear regression of assimilation based on available light response curves and saturating rates of assimilation of CO₂ at the indicated [CO₂]. FIG. 19C. Combined apparent quantum efficiency of photosynthesis ((a) of Bypass 3 determined by linear regression of assimilation based on available light response curves and saturating rates of assimilation of CO₂ at the indicated [CO₂]. Error bars indicated SEM and * indicate significance based on one-way ANOVA P<0.05.

FIG. 20A-20D. Photosynthetic efficiency tested in greenhouse conditions. FIG. 20A. Combined maximum rate of Rubisco carboxylation (Vcmax). FIG. 20B. Combined maximum rate of electron transport (Jmax). Maximum rates of caboxylation and electron transport are modelled from photosynthetic response under changing CO₂ concentration using the PS-Fit model. FIG. 20C. Combined apparent CO₂ compensation point: gamma star (F*) calculated using the common intercept method and slope regression. FIG. 20D. CO₂ assimilation based on internal [CO₂](Ci). Error bars indicate SEM. * indicates statistical difference compared to WT based on one-way ANOVA P values are indicated.

FIG. 21A-21E. Plant productivity and photosynthetic efficiency from the 2017 field trial. FIG. 21A. Percent difference in combined leaf (left bar), stem (middle bar), and total (right bar) biomass compared to WT control for Bypass 3 with and without the PLGG1 RNAi module. Letter indicates statistical differences based on two-way ANOVA P<0.05. FIG. 21B. Total combined accumulated leaf starch for indicated lines. FIG. 21C. Combined apparent quantum efficiency of photosynthesis ((Da) determined by linear regression of assimilation based on available light response curves. FIG. 21D. Combined accumulated assimilation of C02 (A′) based on diurnal analysis of photosynthesis. FIG. 21E. Combined accumulated electron used in electron transport determined from assimilation based on diurnal photosynthesis. Error bars indicate SD and P values are indicated based on ANOVA analysis.

FIG. 22. Knock-down of PLGG1 by RNAi leads to increases Fv′/Fm′ after shift from elevated CO₂ to ambient air. Combined values of 5 transgenic positive plants expressing only the PLGG1 RNAi module compared to trans-gene negative plants from the same TO transformation event. Fv′/Fm′ was measured 3 days after transition from elevated CO₂ to ambient air. Error bars indicate standard deviation.

FIG. 23. Photorespiration bypass results in increased biomass under greenhouse conditions. % difference in total dry weight biomass of the indicated plant lines. EV, empty vector; AP1, Bypass 1; AP2, Bypass 2; AP3, Bypass 3. * indicates statistical difference based on one-way ANOVA. Error bars are SEM.

### DETAILED DESCRIPTION

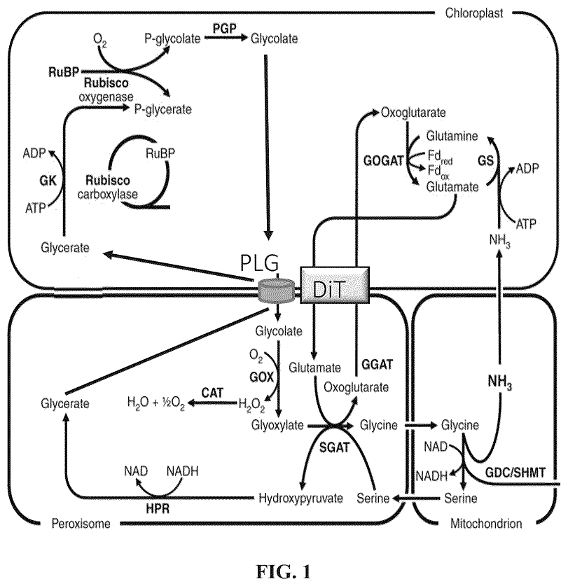

Photorespiration is an energy intensive process that recycles the toxic metabolite 2-phosphoglycolate, a product of RubisCO oxygenation reactions. The photorespiratory pathway is highly compartmentalized involving the chloroplast, peroxisome, cytosol and mitochondria (FIG. 1). Though the soluble enzymes involved in photorespiration are well characterized very few membrane transporters involved in photorespiration have been identified to date. Under photorespiratory conditions Arabidopsis T-DNA insertions targeting the Bile Acid Sodium Symporter Bass6 inhibited photosynthesis and resulted in an ambient air slow growth phenotype that was rescued at elevated CO₂. In addition, metabolite analysis and genetic complementation of glycolate transport in yeast showed that BASS6 was capable of glycolate transport consistent with its involvement in the photorespiratory export of glycolate from Arabidopsis chloroplasts. A double knockout Arabidopsis line including Bass6 and the glycolate/glycerate transporter Plgg1 (bass6-1-plgg1-1) resulted in an additive growth defect, an increase in glycolate accumulation, and reductions in photosynthetic rates compared to either single mutation alone. The data indicate that BASS6 is a chloroplast inner envelope membrane localized transporter of glycolate and exogenous expression of Bass6 can complement the photorespiration mutant phenotype. Knowing the transporters that are responsible for glycolate export from the chloroplasts of C3 plants is critical information in designing strategies to introduce a more energetically efficient photorespiratory pathway thereby improving photosynthetic efficiency.

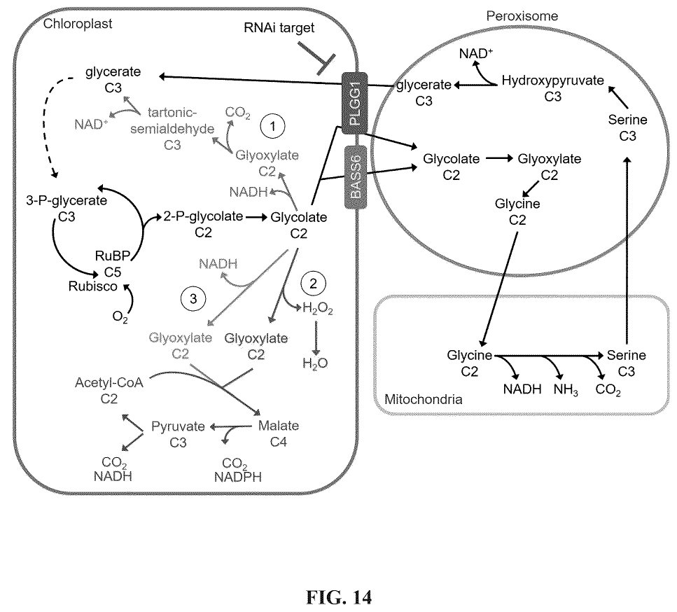

With multiple potential designs that can bypass photorespiration, computer modelling suggests that optimized expression of non-native genes and flux through the bypass pathway are needed to maximize the benefits to crop plants under field conditions. Additionally, reducing or shutting down the native photorespiratory pathway would further increase the benefits of expressing a photorespiratory bypass pathway in plants. We hypothesized that using a synthetic biology approach to generate a library of gene constructs expressing different photorespiratory bypass strategies, while reducing the transport of glycolate from the chloroplast, could provide insight into the benefits of photorespiration bypass and be used to design elite performing plant lines to increase crop productivity (FIG. 14).

Thus, presented herein are also plants containing a recombinant dsRNA (SEQ ID NO: 46) that leads to RNAi knockdown of PLGG1 protein production in combination with expression of bypass pathways. It is expected that either knockdown or knockout strains function similarly given the data showing similar results between such strains. In some embodiments, therefore, plants without a functional PLGG1 protein are combined with Bypass 3 proteins (malate synthase and glycolate dehydrogenase from C. reinhardtii) to produce plants with increased photosynthetic efficiency. Other embodiments provide plants expressing Bypass 3 proteins and lacking a functional BASS6 protein. Transgenic plants expressing Bypass 3 proteins and lacking both a functional BASS6 protein and a functional PLGG1 protein (via knockout or knockdown) are further contemplated herein.

Preferred embodiments of the present disclosure are shown and described herein. It will be obvious to those skilled in the art that such embodiments are provided by way of example only. Numerous variations, changes, and substitutions will occur to those skilled in the art without departing from the disclosure. Various alternatives to the embodiments of the disclosure described herein may be employed in practicing the disclosure. It is intended that the included claims define the scope of the disclosure and that methods and structures within the scope of these claims and their equivalents are covered thereby.

Technical and scientific terms used herein have the meanings commonly understood by one of ordinary skill in the art to which the instant disclosure pertains, unless otherwise defined. Reference is made herein to various materials and methodologies known to those of skill in the art. Standard reference works setting forth the general principles of recombinant DNA technology include Sambrook et al., “Molecular Cloning: A Laboratory Manual”, 2d ed., Cold Spring Harbor Laboratory Press, Plainview, N.Y., 1989; Kaufman et al., eds., “Handbook of Molecular and Cellular Methods in Biology and Medicine”, CRC Press, Boca Raton, 1995; and McPherson, ed., “Directed Mutagenesis: A Practical Approach”, IRL Press, Oxford, 1991. Standard reference literature teaching general methodologies and principles of fungal genetics useful for selected aspects of the disclosure include: Sherman et al. “Laboratory Course Manual Methods in Yeast Genetics”, Cold Spring Harbor Laboratory, Cold Spring Harbor, N.Y., 1986 and Guthrie et al., “Guide to Yeast Genetics and Molecular Biology”, Academic, New York, 1991.

Any suitable materials and/or methods known to those of skill can be utilized in carrying out the instant disclosure. Materials and/or methods for practicing the instant disclosure are described. Materials, reagents and the like to which reference is made in the following description and examples are obtainable from commercial sources, unless otherwise noted.

As used in the specification and claims, use of the singular “a”, “an”, and “the” include plural references unless the context clearly dictates otherwise.

The terms isolated, purified, or biologically pure as used herein, refer to material that is substantially or essentially free from components that normally accompany the referenced material in its native state.

The term “about” is defined as plus or minus ten percent of a recited value. For example, about 1.0 g means 0.9 g to 1.1 g and all values within that range, whether specifically stated or not.

The term “gene” refers to a DNA sequence involved in producing a RNA or polypeptide or precursor thereof. The polypeptide or RNA can be encoded by a full-length coding sequence or by intron-interrupted portions of the coding sequence, such as exon sequences.

The term “primer” refers to an oligonucleotide, which is capable of acting as a point of initiation of synthesis when placed under conditions in which primer extension is initiated. An oligonucleotide “primer” may occur naturally, as in a purified restriction digest or may be produced synthetically.

A primer is selected to be “substantially complementary” to a strand of specific sequence of the template. A primer must be sufficiently complementary to hybridize with a template strand for primer elongation to occur. A primer sequence need not reflect the exact sequence of the template. For example, a non-complementary nucleotide fragment may be attached to the 5′ end of the primer, with the remainder of the primer sequence being substantially complementary to the strand. Non-complementary bases or longer sequences can be interspersed into the primer, provided that the primer sequence is sufficiently complementary with the sequence of the template to hybridize and thereby form a template primer complex for synthesis of the extension product of the primer.

For the purpose of this disclosure, the “sequence identity” of two related nucleotide or amino acid sequences, expressed as a percentage, refers to the number of positions in the two optimally aligned sequences which have identical residues (×100) divided by the number of positions compared. A gap, i.e., a position in an alignment where a residue is present in one sequence but not in the other is regarded as a position with non-identical residues. The alignment of the two sequences is performed by the Needleman and Wunsch algorithm (Needleman and Wunsch, J Mol Biol, (1970) 48:3, 443-53). A computer-assisted sequence alignment can be conveniently performed using a standard software program such as GAP which is part of the Wisconsin Package Version 10.1 (Genetics Computer Group, Madison, Wisconsin, USA) using the default scoring matrix with a gap creation penalty of 50 and a gap extension penalty of 3.

The terms “identical” or percent “identity”, and grammatical variations thereof, in the context of two or more polynucleotides or polypeptide sequences, refer to two or more sequences or sub-sequences that are the same or have a specified percentage of nucleotides or amino acids (respectively) that are the same (e.g., 80%, 85% identity, 90% identity, 99%, or 100% identity), when compared and aligned for maximum correspondence over a designated region as measured using a sequence comparison algorithm or by manual alignment and visual inspection.

The phrase “high percent identical” or “high percent identity”, and grammatical variations thereof in the context of two polynucleotides or polypeptides, refers to two or more sequences or sub-sequences that have at least about 80%, identity, at least about 81%, 82%, 83%, 84%, 85%, 86%, 87%, 88%, 89%, 90%, 91%, 92%, 93%, 94%, 95%, 96%, 97%, 98%, 99%, or 100% nucleotide or amino acid identity, when compared and aligned for maximum correspondence, as measured using a sequence comparison algorithm or by visual inspection. In an exemplary embodiment, a high percent identity exists over a region of the sequences that is at least about 16 nucleotides or amino acids in length. In another exemplary embodiment, a high percent identity exists over a region of the sequences that is at least about 50 nucleotides or amino acids in length. In still another exemplary embodiment, a high percent identity exists over a region of the sequences that is at least about 100 nucleotides or amino acids or more in length. In one exemplary embodiment, the sequences are high percent identical over the entire length of the polynucleotide or polypeptide sequences.

The term “BASS6”, and capitalization and italicized versions thereof, refer to the plant gene and protein, as described herein. In some embodiments, this term may refer to the A. thaliana gene and protein, as described herein. In other embodiments, this term may refer to one or more homologs or orthologs of the gene and protein of any C3 plant. In some embodiments, this term may refer to one or more paralogs of the gene and protein of any C3 plant. In some embodiments, the C3 plant is rice, soybean, potato, cowpea, barley, wheat, or cassava. SEQ ID NO: 1 provides the genomic sequence of the A. thaliana BASS6 gene. SEQ ID NO:2 provides the cDNA sequence of the A. thaliana BASS6 gene. SEQ ID NO: 3 provides the A. thaliana Bass6 protein. When indicated with all lower-case letters in italics, the mutant (e.g., knockout) version of the gene/protein is intended. In A. thaliana, the mutant version may be a single gene/protein. In other C3 plants, the mutant version may be one, some, or all homologs, orthologs, and/or paralogs of the genes/proteins.

The term “PLGG1”, and capitalization and italicized versions thereof, refer to the plant gene and protein, as described herein. In some embodiments, this term may refer to the A. thaliana gene and protein, as described herein. In other embodiments, this term may refer to one or more homologs or orthologs of the gene and protein of any C3 plant. In some embodiments, this term may refer to one or more paralogs of the gene and protein of any C3 plant. In some embodiments, the C3 plant is rice, soybean, potato, cowpea, barley, wheat, or cassava. SEQ ID NO: 4 provides the A. thaliana genomic sequence of the PLGG1 gene. SEQ ID NO:5 provides the cDNA sequence of the A. thaliana PLGG1 gene. SEQ ID NO: 6 provides the A. thaliana Plgg1 protein. When indicated with all lower-case letters in italics, the mutant (e.g., knockout) version of the gene/protein is intended. In A. thaliana, the mutant version may be a single gene/protein. In other C3 plants, the mutant version may be one, some, or all homologs, orthologs, and/or paralogs of the genes/proteins. SEQ ID NO: 46 provides a portion of the PLGG1 coding sequence utilized for RNAi knockdown via production of dsRNA in some transgenic plants of the present invention.

Unless otherwise specifically indicated, the term “CmMS” or “MS”, refers to the Cucurbita maxima malate synthase gene and protein. SEQ ID NO: 43 provides the malate synthase protein (amino acid residues 41-607) fused with the rubisco small subunit signal peptide (amino acid residues 1-40). SEQ ID NO: 42 provides a DNA sequence encoding this protein with the signal peptide and used to produce the Bypass 3 plants described herein. Variants of these nucleic acid and protein sequences, such as DNA encoding proteins with 95% or higher identity to SEQ ID NO: 43 and proteins utilizing alternate signal peptide sequences, are included.

The term “CrGDH” or “GDH”, refer to the Chladymonas reinhardtii glycolate dehydrogenase gene and protein. SEQ ID NO: 45 provides the malate synthase protein (amino acid residues 41-1136) fused with the rubisco small subunit signal peptide (amino acid residues 1-40). SEQ ID NO: 44 provides a DNA sequence encoding this protein with the signal peptide and used to produce the Bypass 3 plants described herein. Variants of these nucleic acid and protein sequences, such as DNA encoding proteins with 95% or higher identity to SEQ ID NO: 45 and proteins utilizing alternate signal peptide sequences, are included.

The term “Bypass” as used herein, refers to a transgenic enzyme pathway introduced into and expressed by a recombinant plant cell. Three Bypass pathways—1, 2, and 3—are shown in Table 1. These are further detailed in the Examples section below.

TABLE 1
Bypass 1, Bypass 2 and Bypass 3 enzymes

| | Plasmid-encoded | | Transgenic genes |
|---|---|---|---|
| | enzymes | Source(s) | expressed |
| Bypass | Glycolate carboligase | E. coli | Exemplary genes: |
| Pathway | (Gcl), Tartonic | | KU512948.1, |
| 1 | Semialdehyde reductase | | WP_001415790.1, |
| | (TSR), glycolate | | KU512945.1, |
| | dehydrogenase subunits | | KU512946.1, |
| | D, E, and F (GdD, GdE, | | KU512947.1 |
| | GdF) | | |
| Bypass | Glycolate Oxidase (GO), | A. thaliana | Exemplary genes: |
| Pathway | Malate Synthase | (GOX1), | NM_112302.4, |
| 2 | (CmMS), and Catalase | Cucurbita | HM755991.1, |
| | HPII (CAT) | maxima | M55161.1 |
| | | (MS), E. | |
| | | coli (katE) | |
| Bypass | Malate Synthase | Cucurbita | SEQ ID NO: 42; |
| Pathway | (CmMS), Glycolate | maxima | SEQ ID NO: 44 |
| 3 | Dehydogenase | (MS), C. | |
| | (CrGDH) | reinhardtii | |
| | | (GYD1) | |

“dsRNA” refers to double-stranded RNA that comprises a sense and an antisense portion of a selected target gene (or sequences with high sequence identity thereto so that gene silencing can occur), as well as any smaller double-stranded RNAs formed therefrom by RNAse or dicer activity. Such dsRNA can include portions of single-stranded RNA, but contains at least 19 nucleotides double-stranded RNA. In one embodiment of the disclosure, a dsRNA comprises a hairpin RNA which contains a loop or spacer sequence between the sense and antisense sequences of the gene targeted, preferably such hairpin RNA spacer region contains an intron, particularly the rolA gene intron (Pandolfini et al., 2003, BioMedCentral (BMC) Biotechnology 3:7 (www.biomedcentral.com/1472-6750/3/7)), the dual orientation introns from pHellsgate 11 or 12 (see, WO 02/059294 and SEQ ID NO: 25 and 15 therein) or the pdk intron (Flaveria trinervia pyruvate orthophosphate dikinase intron 2; see WO99/53050). SEQ ID NO: 46 provides an RNA sequence utilized in some embodiments of the present disclosure to knockdown PLGG1 production.

The enzyme names provided in Table 1, glycolate carboligase, 2-hydroxy-3-oxopropionate reductase, tartonic semialdehyde reductase, glycolate dehydrogenase subunits D, E and F, glycolate oxidase, malate synthase, catalase HPII, and glycolate dehydrogenase, refer to categories of enzymes exemplified by the provided enzymes and sequences. These terms include homologs of these enzymes as well as enzymes that can catalyze the same reactions.

The terms “increase growth” and “increase productivity”, and grammatical variations thereof, as used herein refers to an increase in the rate of growth, or size of a plant at a given timepoint, or an enhanced photosynthetic efficiency of a genetically altered plant in comparison to a non-altered plant of the same species.

### Molecular Biological Methods

An isolated nucleic acid is a nucleic acid the structure of which is not identical to that of any naturally occurring nucleic acid. The term therefore covers, for example, (a) a DNA which has the sequence of part of a naturally occurring genomic DNA molecule but is not flanked by both of the coding or noncoding sequences that flank that part of the molecule in the genome of the organism in which it naturally occurs; (b) a nucleic acid incorporated into a vector or into the genomic DNA of a prokaryote or eukaryote in a manner such that the resulting molecule is not identical to any naturally occurring vector or genomic DNA; (c) a separate molecule such as a cDNA, a genomic fragment, a fragment produced by polymerase chain reaction (PCR), or a restriction fragment; and (d) a recombinant nucleotide sequence that is part of a hybrid gene, i.e., a gene encoding a fusion protein. Specifically excluded from this definition are nucleic acids present in mixtures of (i) DNA molecules, (ii) transformed or transfected cells, and (iii) cell clones, e.g., as these occur in a DNA library such as a cDNA or genomic DNA library.

The term recombinant nucleic acids refers to polynucleotides which are made by the combination of two otherwise separated segments of sequence accomplished by the artificial manipulation of isolated segments of polynucleotides by genetic engineering techniques or by chemical synthesis. In so doing one may join together polynucleotide segments of desired functions to generate a desired combination of functions.

In practicing some embodiments of the disclosure disclosed herein, it can be useful to modify the genomic DNA, chloroplast DNA or mitochondrial DNA of a recombinant strain of a host cell to preclude functional expression of one or more target proteins (e.g., BASS6 or PLGG1) and/or introduce genetic elements allowing for the expression of introduced genes. In preferred embodiments, such a host cell is a plant cell.

Modifications intended to preclude functional expression of a target protein or reduced expression or reduced activity of a target protein can involve mutations of the DNA or gene encoding the target protein, including deletion of all or a portion of a target gene, including but not limited to the open reading frame of a target locus, transcriptional regulators such as promoters of a target locus, and any other regulatory nucleic acid sequences positioned 5′ or 3′ from the open reading frame, insertion of premature stop codons in the open reading frame, and insertions or deletions that shift the reading frame leading to premature termination of translation. Such deletional mutations can be achieved using any technique known to those of skill in the art. Reduced levels of the target protein or reduced activity of the target protein may also be achieved with point mutations or insertions in the DNA or gene encoding the target protein. Mutational, insertional, and deletional variants of the disclosed nucleotide sequences and genes can be readily prepared by methods which are well known to those skilled in the art. Techniques used to achieve reduced levels and/or reduced activity of the target protein may include CRISPR/Cas, TALEN, and Zn-finger nuclease. It is well within the skill of a person trained in this art to make mutational, insertional, and deletional mutations which are equivalent in function to the specific ones disclosed herein. Additionally, such modifications to functional protein production can be achieved via protein “knockdown” approaches, such as RNA interference (RNAi) mediated by double-stranded RNA (dsRNA), siRNA, or other techniques known in the art. An RNA molecule that inhibits expression of a target protein can reduce expression of the gene encoding the protein or may reduce translation of the protein.

Where a recombinant nucleic acid is intended for expression, cloning, or replication of a particular sequence, DNA constructs prepared for introduction into a host cell will typically comprise a replication system (i.e. vector) recognized by the host, including the intended DNA fragment encoding a desired polypeptide, and can also include transcription and translational initiation regulatory sequences operably linked to the polypeptide-encoding segment. Additionally, such constructs can include cellular localization signals (e.g., chloroplast localization signals). In preferred embodiments, such DNA constructs are introduced into a host cell's genomic DNA, chloroplast DNA or mitochondrial DNA.

In some embodiments, a non-integrated expression system can be used to induce expression of one or more introduced genes. Expression systems (expression vectors) can include, for example, an origin of replication or autonomously replicating sequence (ARS) and expression control sequences, a promoter, an enhancer and necessary processing information sites, such as ribosome-binding sites, RNA splice sites, polyadenylation sites, transcriptional terminator sequences, and mRNA stabilizing sequences. Signal peptides can also be included where appropriate from secreted polypeptides of the same or related species, which allow the protein to cross and/or lodge in cell membranes, cell wall, or be secreted from the cell.

Selectable markers useful in practicing the methodologies of the disclosure disclosed herein can be positive selectable markers. Typically, positive selection refers to the case in which a genetically altered cell can survive in the presence of a toxic substance only if the recombinant polynucleotide of interest is present within the cell. Negative selectable markers and screenable markers are also well known in the art and are contemplated by the present disclosure. One of skill in the art will recognize that any relevant markers available can be utilized in practicing the inventions disclosed herein.

Screening and molecular analysis of recombinant strains of the present disclosure can be performed utilizing nucleic acid hybridization techniques. Hybridization procedures are useful for identifying polynucleotides, such as those modified using the techniques described herein, with sufficient homology to the subject regulatory sequences to be useful as taught herein. The particular hybridization techniques are not essential to the subject disclosure. As improvements are made in hybridization techniques, they can be readily applied by one of skill in the art. Hybridization probes can be labeled with any appropriate label known to those of skill in the art. Hybridization conditions and washing conditions, for example temperature and salt concentration, can be altered to change the stringency of the detection threshold. See, e.g., Sambrook et al. (1989) vide infra or Ausubel et al. (1995) Current Protocols in Molecular Biology, John Wiley & Sons, NY, N.Y., for further guidance on hybridization conditions.

Additionally, screening and molecular analysis of genetically altered strains, as well as creation of desired isolated nucleic acids can be performed using Polymerase Chain Reaction (PCR). PCR is a repetitive, enzymatic, primed synthesis of a nucleic acid sequence. This procedure is well known and commonly used by those skilled in this art (see Mullis, U.S. Pat. Nos. 4,683,195, 4,683,202, and 4,800,159; Saiki et al. (1985) Science 230:1350-1354). PCR is based on the enzymatic amplification of a DNA fragment of interest that is flanked by two oligonucleotide primers that hybridize to opposite strands of the target sequence. The primers are oriented with the 3′ ends pointing towards each other. Repeated cycles of heat denaturation of the template, annealing of the primers to their complementary sequences, and extension of the annealed primers with a DNA polymerase result in the amplification of the segment defined by the 5′ ends of the PCR primers. Because the extension product of each primer can serve as a template for the other primer, each cycle essentially doubles the amount of DNA template produced in the previous cycle. This results in the exponential accumulation of the specific target fragment, up to several million-fold in a few hours. By using a thermostable DNA polymerase such as the Taq polymerase, which is isolated from the thermophilic bacterium Thermus aquaticus, the amplification process can be completely automated. Other enzymes which can be used are known to those skilled in the art.

Nucleic acids and proteins of the present disclosure can also encompass homologues of the specifically disclosed sequences. Homology (e.g., sequence identity) can be 50%-100%. In some instances, such homology is greater than 80%, greater than 85%, greater than 90%, or greater than 95%. The degree of homology or identity needed for any intended use of the sequence(s) is readily identified by one of skill in the art. As used herein percent sequence identity of two nucleic acids is determined using an algorithm known in the art, such as that disclosed by Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the NBLAST and XBLAST programs of Altschul et al. (1990) J. Mol. Biol. 215:402-410. BLAST nucleotide searches are performed with the NBLAST program, score=100, wordlength=12, to obtain nucleotide sequences with the desired percent sequence identity. To obtain gapped alignments for comparison purposes, Gapped BLAST is used as described in Altschul et al. (1997) Nucl. Acids. Res. 25:3389-3402. When utilizing BLAST and Gapped BLAST programs, the default parameters of the respective programs (NBLAST and XBLAST) are used. See www.ncbi.nih.gov.

Preferred host cells are plant cells. Recombinant host cells, in the present context, are those which have been genetically modified to contain an isolated nucleic molecule, contain one or more deleted or otherwise non-functional genes normally present and functional in the host cell, or contain one or more genes to produce at least one recombinant protein. The nucleic acid(s) encoding the protein(s) of the present disclosure can be introduced by any means known to the art which is appropriate for the particular type of cell, including without limitation, transformation, lipofection, electroporation or any other methodology known by those skilled in the art.

Transgenic Plants and Plant Cells (t-DNA and Chloroplast Expression)

One embodiment of the present disclosure provides a plant or plant cell comprising one or more modified plant genes and/or introduced genes. For example, the present disclosure provides transgenic plants that lack functional expression of genes encoding chloroplast-localized transport proteins BASS6 and/or PLGG1. Additionally, some plants or plant cells provided herein also express non-native genes, such as enzymes encoded by bacteria-derived, plant-derived, and alga-derived genes. Alternately, some plants or plant cells provided herein can express a native gene in such a way as the protein produced is localized to an organelle (e.g., the chloroplast) or other sub-cellular compartment to which it is not naturally localized. Expression of other genetic elements (e.g., dsRNA resulting in knockdown of PLGG1 protein production) is also contemplated and described herein.

Transformation and generation of genetically altered monocotyledonous and dicotyledonous plant cells is well known in the art. See, e.g., Weising, et al., Ann. Rev. Genet. 22:421-477 (1988); U.S. Pat. No. 5,679,558; Agrobacterium Protocols, ed: Gartland, Humana Press Inc. (1995); and Wang, et al. Acta Hort. 461:401-408 (1998). The choice of method varies with the type of plant to be transformed, the particular application and/or the desired result. The appropriate transformation technique is readily chosen by the skilled practitioner.

Any methodology known in the art to delete, insert or otherwise modify the cellular DNA (e.g., genomic DNA and organelle DNA) can be used in practicing the inventions disclosed herein. For example, a disarmed Ti-plasmid, containing a genetic construct for deletion or insertion of a target gene, in Agrobacterium tumefaciens can be used to transform a plant cell, and thereafter, a transformed plant can be regenerated from the transformed plant cell using procedures described in the art, for example, in EP 0116718, EP 0270822, PCT publication WO 84/02913 and published European Patent application (“EP”) 0242246. Ti-plasmid vectors each contain the gene between the border sequences, or at least located to the left of the right border sequence, of the T-DNA of the Ti-plasmid. Of course, other types of vectors can be used to transform the plant cell, using procedures such as direct gene transfer (as described, for example in EP 0233247), pollen mediated transformation (as described, for example in EP 0270356, PCT publication WO 85/01856, and U.S. Pat. No. 4,684,611), plant RNA virus-mediated transformation (as described, for example in EP 0 067 553 and U.S. Pat. No. 4,407,956), liposome-mediated transformation (as described, for example in U.S. Pat. No. 4,536,475), and other methods such as the methods for transforming certain lines of corn (e.g., U.S. Pat. No. 6,140,553; Fromm et al., Bio/Technology (1990) 8, 833-839); Gordon-Kamm et al., The Plant Cell, (1990) 2, 603-618) and rice (Shimamoto et al., Nature, (1989) 338, 274-276; Datta et al., Bio/Technology, (1990) 8, 736-740) and the method for transforming monocots generally (PCT publication WO 92/09696). For cotton transformation, the method described in PCT patent publication WO 00/71733 can be used. For soybean transformation, reference is made to methods known in the art, e.g., Hinchee et al. (Bio/Technology, (1988) 6, 915) and Christou et al. (Trends Biotech, (1990) 8, 145) or the method of WO 00/42207.

In some embodiments of the present disclosure, one or more genes have been “knocked out” of the host plant cell. Typically, this means deletion of all functional copies of the target gene (e.g., two or more copies, depending on the copy number of the target gene). Any modification of the native sequence(s) that results in a failure of the targeted gene or allele to produce a functional protein is included in the term “knockout”. Such modifications include, but are not limited to, missense mutations, nonsense mutations, stop codon mutations, insertional mutations, deletional mutations, frameshift mutations, and splice site mutations.

Transgenic plants of the present disclosure can be used in a conventional plant breeding scheme to produce more transgenic plants with the same characteristics, or to introduce the genetic alteration(s) in other varieties of the same or related plant species. Seeds, which are obtained from the transformed plants, preferably contain the genetic alteration(s) as a stable insert in chromosomal or organelle DNA. Plants comprising the genetic alteration(s) in accordance with the disclosure include plants comprising, or derived from, root stocks of plants comprising the genetic alteration(s) of the disclosure, e.g., fruit trees or ornamental plants. Hence, any non-transgenic grafted plant parts inserted on a transformed plant or plant part are included in the disclosure.

Introduced genetic elements, whether in an expression vector or expression cassette, which result in the expression of an introduced gene will typically utilize a plant-expressible promoter. A ‘plant-expressible promoter’ as used herein refers to a promoter that ensures expression of the genetic alteration(s) of the disclosure in a plant cell. Examples of promoters directing constitutive expression in plants are known in the art and include: the strong constitutive 35S promoters (the “35S promoters”) of the cauliflower mosaic virus (CaMV), e.g., of isolates CM 1841 (Gardner et al., Nucleic Acids Res, (1981) 9, 2871-2887), CabbB-S (Franck et al., Cell (1980) 21, 285-294) and CabbB-JI (Hull and Howell, Virology, (1987) 86, 482-493); promoters from the ubiquitin family (e.g., the maize ubiquitin promoter of Christensen et al., Plant Mol Biol, (1992) 18, 675-689), the gos2 promoter (de Pater et al., The Plant J (1992) 2, 834-844), the emu promoter (Last et al., Theor Appl Genet, (1990) 81, 581-588), actin promoters such as the promoter described by An et al. (The Plant J, (1996) 10, 107), the rice actin promoter described by Zhang et al. (The Plant Cell, (1991) 3, 1155-1165); promoters of the Cassava vein mosaic virus (WO 97/48819, Verdaguer et al. (Plant Mol Biol, (1998) 37, 1055-1067), the pPLEX series of promoters from Subterranean Clover Stunt Virus (WO 96/06932, particularly the S4 or S7 promoter), an alcohol dehydrogenase promoter, e.g., pAdh1S (GenBank accession numbers X04049, X00581), and the TR1′ promoter and the TR2′ promoter (the “TR1′ promoter” and “TR2′ promoter”, respectively) which drive the expression of the F and 2′ genes, respectively, of the T-DNA (Velten et al., EMBO J, (1984) 3, 2723-2730).

Alternatively, a plant-expressible promoter can be a tissue-specific promoter, i.e., a promoter directing a higher level of expression in some cells or tissues of the plant, e.g., in green tissues (such as the promoter of the PEP carboxylase). The plant PEP carboxylase promoter (Pathirana et al., Plant J, (1997) 12:293-304) has been described to be a strong promoter for expression in vascular tissue and is useful in one embodiment of the current disclosure. Alternatively, a plant-expressible promoter can also be a wound-inducible promoter, such as the promoter of the pea cell wall invertase gene (Zhang et al., Plant Physiol, (1996) 112:1111-1117). A ‘wound-inducible’ promoter as used herein means that upon wounding of the plant, either mechanically or by insect feeding, expression of the coding sequence under control of the promoter is significantly increased in such plant. These plant-expressible promoters can be combined with enhancer elements, they can be combined with minimal promoter elements, or can comprise repeated elements to ensure the expression profile desired.

In some embodiments, genetic elements can be used to increase expression in plant cells can be utilized. For example, an intron at the 5′ end or 3′ end of an introduced gene, or in the coding sequence of the introduced gene, e.g., the hsp70 intron. Other such genetic elements can include, but are not limited to, promoter enhancer elements, duplicated or triplicated promoter regions, 5′ leader sequences different from another transgene or different from an endogenous (plant host) gene leader sequence, 3′ trailer sequences different from another transgene used in the same plant or different from an endogenous (plant host) trailer sequence.

An introduced gene of the present disclosure can be inserted in host cell DNA so that the inserted gene part is upstream (i.e., 5′) of suitable 3′ end transcription regulation signals (i.e., transcript formation and polyadenylation signals). This is preferably accomplished by inserting the gene in the plant cell genome (nuclear or chloroplast). Preferred polyadenylation and transcript formation signals include those of the nopaline synthase gene (Depicker et al., J. Molec Appl Gen, (1982) 1, 561-573), the octopine synthase gene (Gielen et al., EMBO J, (1984) 3:835-845), the SCSV or the Malic enzyme terminators (Schunmann et al., Plant Funct Biol, (2003) 30:453-460), and the T-DNA gene 7 (Velten and Schell, Nucleic Acids Res, (1985) 13, 6981-6998), which act as 3′-untranslated DNA sequences in transformed plant cells.

### Protein Homologs

It will be understood by those skilled in the art that various homologs of the particular proteins disclosed herein can be targeted for deletion, or knock-down approaches, or be utilized to create a Bypass pathway. The following are exemplary, but non-limiting, protein homologs to the particular proteins provided in this disclosure that can be utilized to practice embodiments disclosed herein.

BASS6 homologs include the following (species and accession number): A. thaliana (NP_567671.1), A. thaliana (CAA16569.1), A. lyrata (XP_002867746.1), Eutrema salsugineum (XP_006413612.1), Capsella rubella (XP_006282633.1), Camelina sativa (XP_010433982.1), Camelina sativa (XP_010448822.1), Arabis alpine (KFK39256.1), Brassica oleracea var. oleracea (XP_013593377.1), Brassica napus (XP_013737613.1), Brassica rapa (XP_009137288.1), and Raphanus sativus (XP_018484108.1).

PLGG1 homologs include the following (species and accession number): Arabidopsis thaliana (NP_564388.1), Arabidopsis thaliana (AAM65181.1), Arabidopsis lyrata (XP_020868671.1), Arabidopsis lyrata(EFH69957.1), Capsella rubella (XP_006307262.1), Camelina sativa (XP_010478626.1), Camelina sativa (XP_010461027.1), Camelina sativa (XP_010499753.1), Brassica napus (XP_013733826.1), Raphanus sativus (XP_018457661.1), Brassica oleracea var. oleracea (XP_013587088.1), Brassica napus (XP_013731498.1), Brassica rapa (XP_009114919.1), Eutrema salsugineum (XP_006415255.1), Brassica oleracea var. oleracea (XP_013587305.1), Brassica napus (XP_022559249.1), Brassica napus (CDY35540.1), Brassica rapa (XP_009145211.1), Raphanus sativus (XP_018486680.1), Arabis alpine (KFK44969.1), Brassica napus (CDY59206.1), Brassica napus (XP_013731491.1), Brassica napus (CDY22583.1), Tarenaya hassleriana (XP_010518925.1), Ricinus communis (XP_002519004.1), Hevea brasiliensis (XP_021652349.1), Citrus sinensis (XP_006471454.1), Brassica napus (XP_022575243.1), and Juglans regia (XP_018843901.1).

Malate synthase homologs include the following (species and accession number): Cucurbita maxima (XP_023000792.1), Cucurbita pepo (XP_023519701.1), Cucurbita moschata (XP_022923624.1), Momordica charantia (XP_022137538.1), Cucumis sativus (XP_004152519.1), Cucumis melo (XP_008439505.1), Theobroma cacao (EOY22418.1), Juglans regia (XP_018821986.1), Eucalyptus grandis (XP_010037447.1), Eucalyptus grandis (KCW49165.1), Herrania umbratica (XP_021286625.1), Theobroma cacao (XP_007037917.2), Arachis duranensis (XP_020997255.1), Gossypium barbadense (PPR87616.1), Prunus avium (XP_021800964.1), Vitis vinifera (XP_002279452.1), Quercus suber (XP_023901530.1), Quercus suber (POF20494.1), Gossypium raimondii (XP_012468378.1), Cephalotus follicularis (GAV68244.1), Gossypium barbadense (PPD76680.1), Capsicum baccatum (PHT53703.1), Nicotiana tabacum (XP_016464464.1), Capsicum chinense (PHU23662.1), Ricinus communis (XP_002511225.1), Capsicum annuum (XP_016563806.1), Gossypium arboretum (XP_017604788.1), Citrus clementina (XP_006440060.1), Medicago truncatula (XP_013444720.1), Durio zibethinus (XP_022737455.1), Trifolium pretense (PNY13237.1), Medicago truncatula (ACJ85740.1), Nicotiana tomentosiformis (XP_009595632.1), Citrus unshiu (GAY51023.1), Prunus mume (XP_008239432.1), Prunus sibirica (AIU64851.1), Prunus persica (XP_020420471.1), Solanum lycopersicum (XP_004236345.1), Parasponia andersonii (PON64176.1), Ricinus communis (NP_001310646.1), Citrus sinensis (XP_006476991.1), Glycine max (NP_001347240.1), Nicotiana attenuate (XP_019261379.1), Daucus carota subsp. Sativus (XP_017250345.1), Aquilegia coerulea (PIA33657.1), Trema orientalis (PON95652.1), Helianthus annuus (XP_022038983.1), Macleaya cordata (OVA17558.1), Nicotiana sylvestris (XP_009776635.1), Jatropha curcas (XP_012079884.1), Lupinus angustifolius (XP_019463065.1), Vigna angularis (XP_017425062.1), Solanum pennellii (XP_015070973.1), Glycine soja (KHN14088.1), Tarenaya hassleriana (XP_010555537.1), Solanum lycopersicum (XP_010319064.1), Solanum tuberosum (XP_006351486.1), Solanum pennellii (XP_015070972.1), Solanum tuberosum (XP_006351485.1), Cicer arietinum (XP_004510708.1), Vigna radiata var. radiate (XP_022639201.1), Lactuca sativa (XP_023732731.1), Populus euphratica (XP_011024067.1), Populus trichocarpa (PNT01248.1), Carica papaya (XP_021909023.1), Cajanus cajan (XP_020216385.1), Nicotiana tabacum (XP_016435566.1), Olea europaea var. sylvestris (XP_022878435.1), Malus domestica (XP_008374272.1), Olea europaea var. sylvestris (XP_022878436.1), Hevea brasiliensis (XP_021681023.1), Morus notabilis (XP_010103099.1), Punica granatum (OWM90581.1), Arabidopsis thaliana (NP_196006.1), Arabidopsis lyrata (XP_002873119.1), Ipomoea nil (XP_019198829.1), Erythranthe guttata (XP_012854673.1), Eutrema salsugineum (XP_006398845.1), Camelina sativa (XP_010490879.1), Sesamum indicum (XP_011073010.1), Ziziphus jujube (XP_015878765.1), Brassica rapa (XP_009125524.1), Fragaria vesca subsp. Vesca (XP_004297548.1), Camelina sativa (XP_010423656.1), Glycine max (XP_003525685.1), Capsella rubella (XP_006286626.1), Brassica napus (XP_013720273.1), Raphanus sativus (XP_018468682.1), Brassica napus (CDY14170.1), Arabis alpine (KFK24848.1), Brassica oleracea var. oleracea (XP_013621105.1), Solanum tuberosum (XP_006351487.1), Dorcoceras hygrometricum (KZV21744.1), Corchorus capsularis (OM084006.1), Manihot esculenta (OAY30724.1), and Brassica napus (CAA73793.1).

Glycolate hydrogenase homologs include the following (species and accession number): Chlamydomonas reinhardtii (XP_001695381.1), Chlamydomonas reinhardtii (ABG36932.1), Volvox carterif nagariensis (XP_002946459.1), Gonium pectoral (KXZ46746.1), Chlamydomonas eustigma (GAX77289.1), Chlorella variabilis (XP_005852216.1), Coccomyxa subellipsoidea (XP_005648725.1), Micromonas commode (XP_002506446.1), Auxenochlorella protothecoides (XP_011399156.1), Ostreococcus tauri (XP_003074362.2), Ostreococcus lucimarinus (XP_001415862.1), Ostreococcus tauri (OUS42650.1), Bathycoccus prasinos (XP_007511439.1), Micromonas pusilla (XP_003063153.1), Chrysochromulina sp. (KO033603.1), and Guillardia theta (XP_005827919.1).

Having generally described this invention, the same will be better understood by reference to certain specific examples, which are included herein to further illustrate the invention and are not intended to limit the scope of the invention as defined by the claims.

### EXAMPLES

### Example 1: Materials and Methods

### Plant Material and Growth Conditions

A. thaliana Columbia (Col-0) was used as wild type reference. Salk_03569C (plgg1-1), CS859747 (bass6-1), and Salk_052903C (bass6-2) were obtained from the Arabidopsis Biological Resource Center (abrc.osu.edu). Plants were grown in either elevated (2000 ppm CO₂) or ambient (400 ppm CO₂) at 8 h light/16 h dark cycle (22° C./18° C.) at 250 μmol·m⁻²·s⁻¹ PAR and 65% relative humidity (RH) in growth chambers (Conviron, USA) using LC1 Sunshine Mix.

### Chlorophyll Fluorescence Measurements

Arabidopsis plants were grown under ambient air conditions and moved to a sealed clear plastic container at low CO₂ conditions under constant illumination for 24 hours similar to (Badger et al., 2009) before chlorophyll fluorescence measurements. Chlorophyll fluorescence measurements were done as previously described (Oxborough and Baker, Plant, Cell & Environ. (1997) 20:1473-83; Badger et al., supra). Briefly, Fv/Fm images were taken after 15 minutes dark adaptation of 4 week old plants using the CF Imager Technologica (www.technologica.co.uk). Maximum flash intensity was 6800 μmol·m⁻²·s⁻¹ for 800 milliseconds. Image values were obtained for each individual plant by detecting colonies within the fluorimager software program defining each position.

### Cloning

All expression vectors are described in Table 2. Promoters and open reading frames were synthesized based on sequence obtained from The Arabidopsis Information Resource (TAIR). Restriction sites and 4 base pair regions of homology were designed according to common syntax in plant synthetic biology (Patron et al., New Phytol. (2015) 208:13-19). Constructs were then assembled using Golden Gate cloning protocol then subcloned into a binary vector (EC50505) (Werner et al., Bioengineered (2012) 3:38-43; Engler et al., ACS Synth. Biol. (2014) 3:839-43; Marillonnet and Werner, In Glyco-engineering, A. Castilho, ed (Springer New York), pp. 269-84 (2015); Patron et al., supra). For stable transformation the binary vectors were transformed into Agrobacterium tumefaciens C58C1 by electroporation, and then transformed into the Col-0 wild type strain, plgg1-1 or bass6-1 T-DNA insertion lines by floral dip (Clough and Bent, Plant J. (1998) 16:735-43). Transformed lines were selected based on BASTA resistance and gene insertion was verified by PCR analysis. For transient expression, constructs were designed as above driven by the CaMV35s promoter with a C-terminal GFP fusion.

TABLE 2
Plasmids

| Plasmid | Inserted genes | Promoter | Vector | Source |
|---|---|---|---|---|
| EC50505 | none | | EC50505 | ENSA (project |
| | | | | ensa.ac.uk) |
| EC27349 | p19, eGFP | 2 × 35 s | EC50505 | This study |
| EC27357 | p19, BASS6- | 2 × 35 s | EC50505 | This study |
| | eGFP | | | |
| p415 | none | | p415 | ATCC-87374 |
| ADH1 | | | ADH1 | |
| | BASS6 | ADH1 | p415 | This study |
| | | | ADH1 | |
| | PLGG1 | ADH1 | p415 | This study |
| | | | ADH1 | |
| EC15325 | BAR | NOS | EC50505 | ENSA (project |
| | | | | ensa.ac.uk) |
| EC27403 | BAR, PLGG1 | NOS, PLGG1 | EC15325 | This study |
| EC27404 | BAR, BASS6 | NOS, BASS6 | EC15325 | This study |
| EC27406 | BAR, BASS6 | NOS, PLGG1 | EC15325 | This study |

For the transient expression work on isolated protoplasts, the BASS6-GFP construct was cloned as follows. The coding sequence of A. thaliana Bass6 (AT4G22840) was synthesized by GENEWIZ Inc. with a C-terminal tag containing mGFP6 (Haseloff, J., Method Cell Biol. (1999) 58:139-51), 6×HIS and MYC, into a modified gateway-compatible pUC57 plasmid from which it was recombined in pMDC32. The AtPLGG1-GFP construct was previously published (Rolland et al., Front. Plant Sci. (2016) 7:185).

Agro-Infiltration of Nicotiana benthamiana and Microscopy

Growth and infiltration experiments are described in (Rolland et al., supra). Briefly, Agrobacterium tumefaciens GV3101 (pMP90) were transformed with plasmids of interest and grown in LB media containing rifampicin (50 μg/ml) and kanamycin (30 μg/ml). Cultures were grown for about 24 h in a 28-30° C. incubator and used for transformation of N. benthamiana leaves. Bacteria containing P19 (OD600=0.3) were mixed with bacteria containing the plasmid of interest and/or the ER compartment marker (OD600=0.5) (plasmid CD3-959 from the Arabidopsis Biological Resource Center, http://abrc.osu.edu, (Nelson et al., 2007)). Cells were centrifuged for 8 minutes at 2150×g and resuspended (10 mM MES pH 5.6, 10 mM MgCl₂, and 150 μM acetosyringone). The cells were incubated for 2 h at room temperature and infiltrated into 3-4 weeks old N. benthamiana leaves.

Protoplast preparation was completed as described previously (Rolland et al., supra). Two days after infiltration, a 4 cm² area of infiltrated leaf was cut with a scalpel and transferred in a 5 ml syringe in which 2 ml of digestion solution was added and a gentle vacuum was manually applied. The infiltration solution and the leaf tissue was transferred to a 2 ml Eppendorf tube and incubated for 1 h at room temperature. Leaf debris was removed and protoplasts were allowed to sediment before the solution was replaced with imaging solution (0.4 M mannitol, 20 mM KCl, 20 mM MES pH 5.6, 10 mM CaCl₂), 0.1% [w/v] BSA).

Protoplasts were observed and imaged using an upright Zeiss LSM780 confocal laser-scanning microscope (Carl Zeiss), a 40× water immersion objective (NA=1.1) and the Zen 2011 software package (Carl Zeiss). GFP and chlorophyll were excited at 488 nm and recorded at 499-534 nm and at 630-735 nm, respectively. mCherry was excited at 561 nm and recorded at 579-633 nm in a separate track (although not shown in this study).

Sliced whole leaf tissue was visualized using a Light sheet Z1 (Carl Zeiss INC. Oberkochen, Germany) microscope from infiltrated leaf tissue as described, using a 40× objective (NA=1.0) using the Zen light sheet software package (Carl Zeiss). GFP and chlorophyll were excited at 488 nm and emission selection was recorded at 505-545 and 660 nm respectively.

### Metabolic Profiling

For metabolite analysis ˜40 mg of fresh leaf tissue was frozen in liquid nitrogen, crushed and then extracted with 500 μL of 100% methanol. Samples were then submitted to the Metabolomics Center, Roy J. Carver Biotechnology Center, University of Illinois at Urbana-Champaign where two additional extractions were then performed: isopropanol: acetonitrile: water (3:3:2 v/v), and chloroform: methanol (2:2 v/v). Metabolites were analyzed using a GC-MS system (Agilent Inc., CA, USA) consisting of an Agilent 7890 gas chromatograph, an Agilent 5975 mass selective detector and a HP 7683B auto sampler. Gas chromatography was performed on a ZB-5MS (60m×0.32 mm I.D. and 0.25 um film thickness) capillary column (Phenomenex, CA, USA). The inlet and MS interface temperatures were 250° C., and the ion source temperature was adjusted to 230° C. An aliquot of 1 μL was injected with the split ratio of 10:1. The helium carrier gas was kept at a constant flow rate of 2 ml/min. The temperature program was: 5-min isothermal heating at 70° C., followed by an oven temperature increase of 5° C. min⁻¹ to 310° C. and a final 10 min at 310° C. The mass spectrometer was operated in positive electron impact mode (EI) at 69.9 eV ionization energy at m/z 30-800 scan range. The spectra of all chromatogram peaks were compared with electron impact mass spectrum libraries NIST08 (NIST, MD, USA), W8N08 (Palisade Corporation, NY, USA), and a custom-built database (464 unique metabolites). All known artificial peaks were identified and removed. To allow comparison among samples, all data were normalized to the internal standard in each chromatogram and the sample wet weight. The spectra of all chromatogram peaks were evaluated using the AMDIS 2.71 (NIST, MD, USA) program. Metabolite concentrations were reported as concentrations relative to the internal standard (i.e., target compound peak area divided by peak area of hentriacontanoic acid: Nᵢ=Xᵢ*X⁻¹IS) per gram wet weight. The instrument variability was within the standard acceptance limit of 5%.

### Photosynthesis Measurements

The youngest fully expanded leaves of 30-40 day old Arabidopsis plants grown at elevated [CO₂] were used for analysis of photosynthesis by gas exchange. Gas exchange measurements were performed using a Li—COR 6400XT with a 2 cm² fluorescence measuring head with gasket leaks corrected for as outlined in the manual (LI-COR Biosciences, Lincoln, NE, USA). A, gₛ and Cᵢ measurements were obtained at indicated CO₂ concentrations at a leaf temperature of 25° C. and saturating light (1000 μmol·m⁻²·s⁻¹). ACᵢ curves were measured in a range of CO₂ (50-2000 ppm) under the same temperature and light conditions stated above after acclimation under ambient CO₂. V_{c Max}, J_{Max} R_{d} and gₛ were determined using ACᵢ data and the PsFit model (Bernacchi et al., Plant Cell. Environ (2003) 26: 1419-1430).

### RT-PCR

cDNA was generated from RNA extracted using the plant RNeasy extraction kit and Quantitec reverse transcription kit (QIAGEN USA) from 4-week old Arabidopsis plants grown under 8 h/16 h day night cycle at 180 μmol·m⁻²·s⁻¹ PAR and 22° C./18° C. temperature regime at 65% RH. Three biological replicates including three technical replicates each were used for all samples. Samples were analyzed using a Bio-Rad CFX connect real-time PCR system (Bio-Rad laboratories, USA) and relative changes in transcript were determined using the ΔΔCt method using primers directed toward violaxanthin de-epoxidase (VDE), Plgg1, and Bass6 transcripts. cDNA was amplified using a SSO advanced SYBR green master mix (Bio-Rad) and primer sequences are described in Table 3.

TABLE 3
Primers

| Primer name | SEQ ID NO: | Sequence |
|---|---|---|
| p35s seq F | SEQ ID NO: 7 | CTCTCTGCCGACAGTGGT |
| t35s seq R | SEQ ID NO: 8 | CTTATATGCTCAACACATGAGCG |
| Salk_053469 LP | SEQ ID NO: 9 | ATAACCGCGAGATAGAGAGGC |
| Salk_053469RP | SEQ ID NO: 10 | CCCATGGCTACTCTTTTAGCC |
| LBb1.3 | SEQ ID NO: 11 | ATTTTGCCGATTTCGGAAC |
| Plgg1-005F | SEQ ID NO: 12 | GCCGGATCCATGGCTTCGTGCTC |
| | | TAAGATCCGTTTCGGT |
| Plgg1-006R | SEQ ID NO: 13 | GCCCTCGAGTCAGCCGACGACCG |
| | | CTAGC |
| Bass6-001F | SEQ ID NO: 14 | GCTCTAGAATGAGCGTGATCACA |
| | | ACTCC |
| Bass6-002R | SEQ ID NO: 15 | GACTCGAGTTAAAATGTGTTACT |
| | | CTTTTC |
| Plgg1-RT 1F | SEQ ID NO: 16 | CTACTCTTTTAGCCACTCCTATC |
| | | TTC |
| Plgg1-RT 2R | SEQ ID NO: 17 | AGATTCAACTTCTGGGCACC |
| Bass6-RT 1F | SEQ ID NO: 18 | TCGCAGTCAACGGATTCAAG |
| Bass6-RT-2R | SEQ ID NO: 19 | TCTACGCCACAAATTCCTCG |
| VDE-RT-1F | SEQ ID NO: 20 | TGAGTTCAACGAGTGTGCTG |
| VDR-RT-2R | SEQ ID NO: 21 | ACTTGTAATGTACCACTTCCCG |

### Yeast Complementation and Glycolate Uptake

Yeast plasmids were assembled as previously described (South et al., J. Biol. Chem. (2010) 285:595-607). Briefly, RNA was obtained from Col-0 wild-type Arabidopsis and converted to cDNA by RNeasy extraction kit and Quantitec reverse transcription kit (QIAGEN, Hilden, Germany). The CDS sequence of full length Bass6 and Plgg1 minus the chloroplast localization signal (1-24) were amplified by PCR using primers described in Table 3. The PCR product was cloned into the pRS415-ADH1 vector (ATCC, VA USA) using BamHI and XhoI restriction sites. Yeast transformations were performed as previously described with BY4741 mat a wild-type and ady2Δ strains (GE Dharmacon) (South et al., supra; South et al., Proc. Nat'l Acad. Sci. USA (2013) 110:E1016-E1025).

Complementation of the ady2Δ strain was performed by comparing growth analysis using glycolate as a carbon source. Wild-type and ady2Δ strains transformed with Bass6, Plgg1, or empty vector expression plasmids were grown in 50 mL cultures in synthetic complete media lacking leucine (SC-Leu) until reaching an optical density of OD₆₀₀ between 0.6 and 0.8. Cells were then washed in water twice and resuspended in water. A spot assay was performed on SC-Leu plates containing 2% glucose, lactate, or glycolate as a carbon source. 5 uL spots were dropped onto plates with five 10-fold serial dilutions starting with an OD₆₀₀ of 0.1. Plates were then incubated at 30° C. Photographs of plates were taken at Id (glucose), 2d (lactate), 7d (glycolate).

Glycolate uptake measurements were performed similarly to glycerol uptake describe previously (Oliveira et al., FEMS Microbiol. Lett. (1996) 142:147-53). Yeast strains were grown in 50 mL cultures in SC-Leu at 30° C. until reaching an optical density of OD₆₀₀ between 0.6 and 0.8. Cells were harvested and washed twice with water and resuspended in 100 mM Tris/citrate buffer pH 5.0 at a concentration of 30 mg/ml dry weight. After 2 minute incubation at 25° C. the reaction was started with the addition of 150 μL of (SC-Leu/glycolate) containing 1 μL aqueous solution of 50 mCi/mmol (3.7*10\hat{ }3 [Bq] total) [¹⁴C]-glycolic acid (American Radiolabeled chemicals, MO USA). After 10 minutes the reaction was stopped by the addition of 5 mL of ice cold water. The reaction mixtures were then filtered on glass fiber filters (Fisher Scientific USA) and washed 3 times with 5 mL of ice-cold water. [¹⁴C]-glycolate uptake was measured by scintillation using the filters plus 4 mL of scintillation fluid (RPI Bio-safe II) using a Packard Tri-Carb liquid scintillation counter (Perkin Elmer USA).

### Statistical Analyses

All experiments had at least 3 biological replicates and data indicate the average values. Relative growth analysis and relative changes in mRNA levels include standard deviation and significance using a student's T-test. Metabolite analysis and photosynthetic measurements were analyzed either by a one-way ANOVA (genotype) or two-way ANOVA (genotype by CO₂ treatment) with a significance threshold of P<0.05. All ANOVA were followed with a Tukey's post-hoc test and determined using statistical software (OriginPro 9.1, OriginLab, MA USA).

### Example 2: Analysis of Arabidopsis Bass6 Mutant Phenotypes

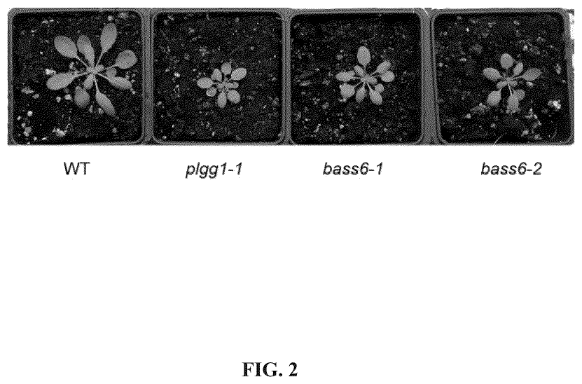

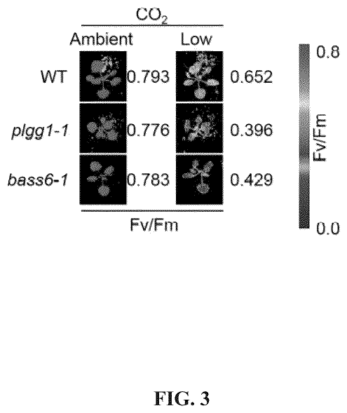

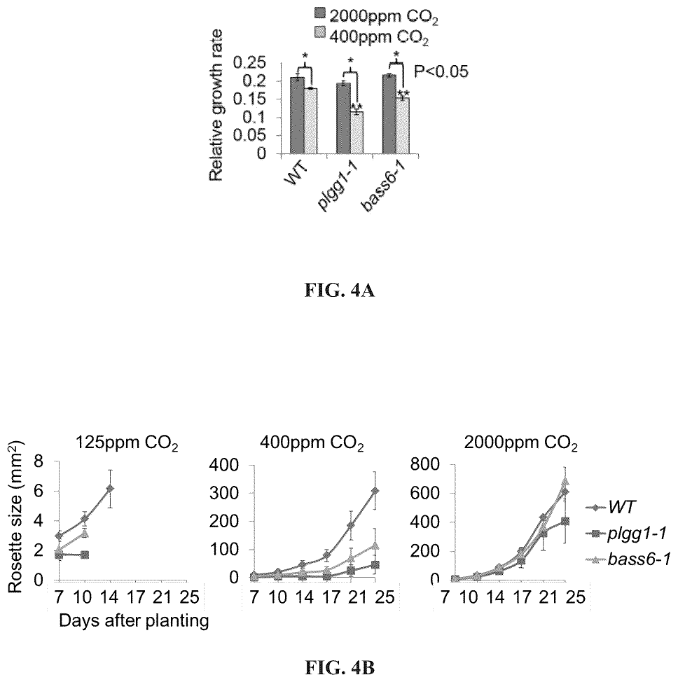

Two independent T-DNA insertion lines targeting the gene At4g22840, which lack the expression of the putative chloroplast inner membrane protein BASS6 were analyzed. To determine if BASS6 is involved in photorespiration, the two T-DNA lines (bass6-1 and bass6-2) were grown under ambient CO₂ (400 ppm) conditions. Compared to the wild type control, both bass6 mutant lines exhibited a smaller rosette size similar to that of the glycolate/glycerate transporter mutant involved in photorespiration plgg1-1 (FIG. 2—representative photos of bass6 and plgg1 mutants compared to WT grown at ambient CO₂ (8 weeks 400 ppm CO₂ at 8 h light/16 h dark cycle (22° C./18° C.) at 250 μmol·m⁻²·s⁻¹ light intensity in growth chambers)). Illumination of photorespiratory mutants under low concentrations of CO₂ resulted in reduced dark-adapted Fv/Fm chlorophyll fluorescence potentially due to photodamage to photosystem II (Badger et al., supra). Both plgg1-1 and bass6-1 lines showed a significant reduction in Fv/Fm when compared to wild type following 24 hours under constant illumination, though no reduction in Fv/Fm was observed prior to low [CO₂] treatment (FIG. 3). To verify the photorespiratory mutant phenotype, growth analysis of the bass6-1 mutant was performed at low, ambient, and elevated CO₂. Consistent with a classical photorespiratory mutant phenotype both the bass6-1 and the plgg1-1 mutants failed to grow at 125 ppm CO₂ (FIG. 4B). Under ambient CO₂ conditions both the bass6-1 and the plgg1-1 T-DNA lines exhibited a slow growth phenotype when compared to the wild type control (FIGS. 4A and 4B). Importantly, the slow growth phenotype was recovered to the wild type phenotype in both the bass6-1 and plgg1-1 mutants when grown in high [CO₂] conditions (FIGS. 4A and 4B).

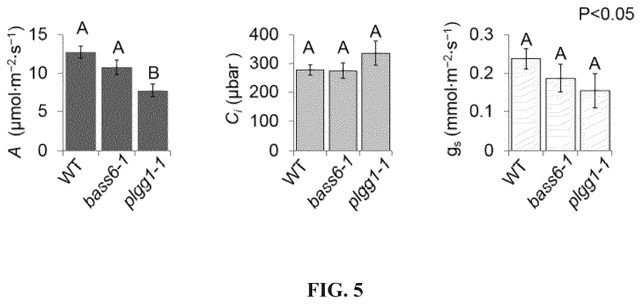

Mutants in the photorespiratory pathway often require high levels of CO₂ for wild-type or near wild-type growth, which are conditions that the RubisCO oxygenation reaction is suppressed to very low levels (Timm and Bauwe, supra). At ambient [CO₂] photorespiration mutants commonly show a reduction in photosynthesis characterized by reductions in carbon assimilation (A), Rubisco V_{c max} and Jₘₐₓ parameters. Similar to previous reports (Pick et al., supra; Walker et al., Photosyn Res. (2016) 129:93-103), plgg1-1 exhibited a lower photosynthetic rate compared to wild type (FIG. 5). The bass6-1 plants also showed a slight reduction in photosynthesis at ambient [CO₂] compared to WT with no detectable changes in internal CO₂ concentration (Cᵢ) or stomatal conductance (gₛ) (FIG. 5). To evaluate the biochemical limitations to photosynthesis of bass6-1 and plgg1-1 lines, the response of carbon assimilation (A) on the intercellular [CO₂] within the leaf (Cᵢ) was investigated. Consistent with the single point photosynthetic measurements, both bass6-1 and plgg1-1 had decreased V_{c max} and Jₘₐₓ values (Table 3). For Table 4, letters indicate statistical difference based on ANOVA analysis p≤0.5. V_{c}Max, maximum carboxylation rate allowed by Rubisco; J Max, maximum rate of photosynthetic electron transport; R_{d}, day respiration; gₛ, stomatal conductance. The reduction in the rate of photosynthesis in the bass6-1 line was not as large as in the plgg1-1 mutant, which is consistent with comparative rosettes sizes and the growth rates of the bass6-1 vs plgg1-1 mutant plants (compare FIG. 2 and FIG. 4A with FIG. 5 and Table 4).

TABLE 4
Photosynthetic parameters based on ACᵢ data using PsFit model.

| | V_{_{c}}_{Max} 25° C. | J_{Max} 25° C. | R_{d} | gₛ |
|---|---|---|---|---|
| | (μmol · | (μmol · | (μmol · | (mmol · |
| | m⁻² · s⁻¹) | m⁻² · s⁻¹) | m⁻² · s⁻¹) | m⁻² · s⁻¹) |
| Col-0 | 58.64^{A} ± 0.93 | 121.98^{A} ± 1.61 | 1.44^{A} ± 0.16 | 0.24^{A} ± 0.04 |
| WT | | | | |
| bass6-1 | 49.60^{B} ± 1.62 | 107.38^{B} ± 1.18 | 1.20^{AB} ± 0.33 | 0.22^{A} ± 0.05 |
| plgg1-1 | 38.37^{C} ± 3.12 | 97.33^{C} ± 2.18 | 2.24^{B} ± 0.5 | 0.16^{A} ± 0.04 |

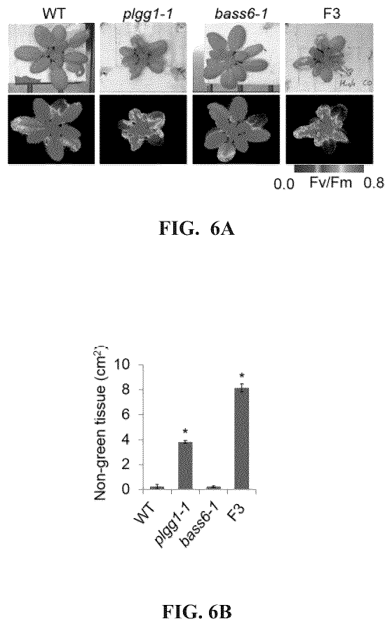

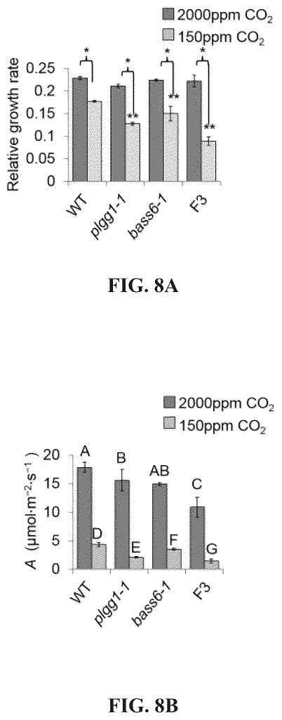

Previous characterization of plgg1-1 demonstrated that when plgg1-1 mutants develop chlorotic lesions on their leaves when grown at high levels of CO₂ and then shifted to ambient air (Pick et al., supra). Using chlorophyll fluorescent Fv/Fm detection, chlorotic lesions are detectable after 3 days in ambient air conditions. Consistent with previous studies the plgg1-1 mutant develops lesions on leaves after shift to ambient air (FIG. 6A). While the bass6-1 mutant alone does not develop observable chlorotic lesions on leaves after shift to ambient CO₂ at three days or as long as 7 days after transfer (FIG. 6A), the homozygous F3 generation of the bass6-1 and plgg1-1 cross develops chlorotic lesions more severely when compared to the plgg1-1 line alone consistent with an additive photorespiratory mutant phenotype (FIG. 6A and FIG. 6B). With an observed doubling in severity of chlorotic lesions in the bass6, plgg1 double mutant we hypothesized there would also be an additive growth defect and further reductions in photosynthetic rates. As predicted, the growth phenotype observed in the F3 double mutant plants show a further decrease, and the reduction in photosynthetic rate appears to be additive when both PLGG1 and BASS6 functions are missing (FIG. 8A and FIG. 8B).

### Example 3: BASS6 Protein is Localized in the Chloroplast Envelope

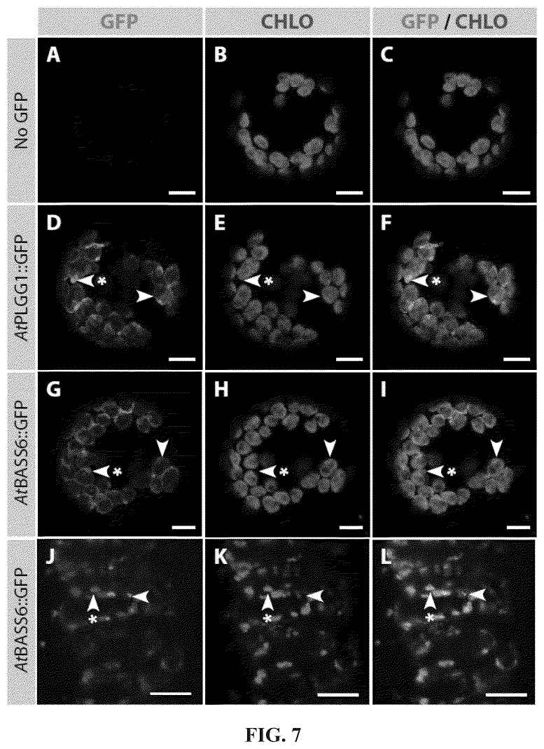

Although a previous study suggested that BASS6 protein localizes to the chloroplast envelope, subcellular location prediction programs more strongly favored mitochondrial localization of BASS6 (Gigolashvili et al., supra). To determine the localization of BASS6 protein, transient expression of BASS6-GFP fusion proteins was analyzed in both protoplasts and whole leaf tissue in Nicotiana benthamiana. Chlorophyll auto-fluorescence was used to identify chloroplasts (FIG. 7, panels B, E, H, and K). The BASS6-GFP signal surrounded chloroplast autofluorescence (FIG. 7, panels G-I), similarly to the known glycolate/glycerate transporter PLGG1 (FIG. 7, panels D-F), indicating that BASS6 is localized to the chloroplast envelope. Furthermore, expression of either BASS6-GFP or PLGG1-GFP induced the formation of stromules (starred arrowheads in FIG. 7, panels D-I) which shapes are typical of proteins localized in the chloroplast inner envelope membrane (Breuers et al., Frontiers Plant Sci., (2012) 3:7). Light-sheet microscopy experiments from whole leaf tissue also showed localization to the chloroplast envelope and additional non-chloroplastic regions, though this may have resulted from transient overexpression (FIG. 7, panels J-L). As an additional control, a GFP control protein was compared to BASS6-GFP showing that BASS6 does not localize to the cytosol.

### Example 4: Analysis of Bass6-1 Metabolite Profiles

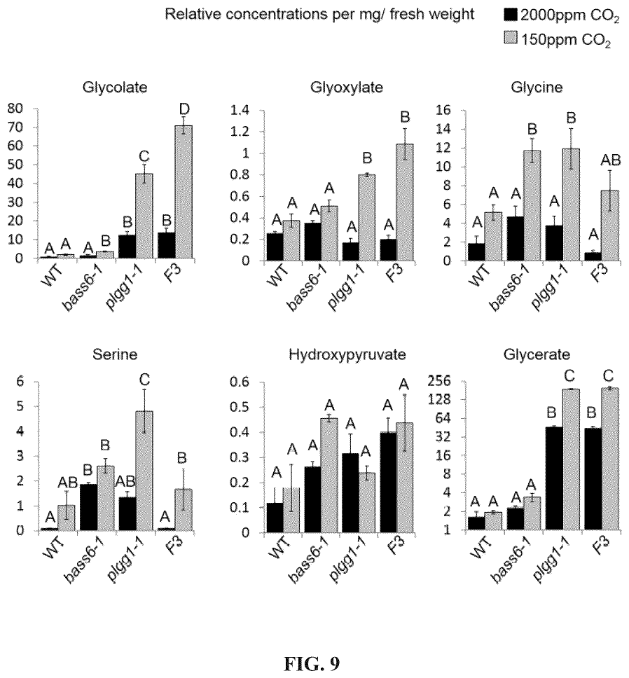

When RubisCO oxygenation rates are appreciable, mutant plants that have defects in photorespiration accumulate various metabolite intermediates within the photorespiratory pathway. To help identify the transport step of BASS6 in the photorespiration pathway, metabolite profiles were analyzed in leaf tissue exposed to either high (2000 ppm) or low (150 ppm) [CO₂]. When compared to the wild type control under high [CO₂] conditions the bass6-1 line showed an increase in the amino acid serine, whereas the previously reported plgg1-1 line accumulated multiple photorespiratory intermediates such as glycolate, glycine and glycerate, even at high CO₂ concentrations (FIG. 9). When leaves were exposed to low levels of CO₂ to increase the RubisCO oxygenation rate, the levels of glycine and glycolate in bass6-1 were significantly increased compared to wild type (FIG. 9). As a comparison, bass6-1 accumulated glycine levels similar to the plgg1-1 plants when leaves were exposed to low levels of CO₂ (FIG. 9).

To determine if the combined loss of BASS6 and PLGG1 led to an additive increase in level of photorespiratory intermediates, the homozygous F3 cross between the plgg1-1 and the bass6-1 lines was compared to wild type and the respective single mutants. The F3 double mutant exhibits a significant increase in glycolate accumulation when compared to wild type and the single mutant lines, and increases in other intermediates such as glycerate similar to the plgg1-1 single mutant (FIG. 9). The metabolite profile data show that the loss of BASS6 function can result in the accumulation of photorespiratory metabolites. In addition, loss of both BASS6 and PLGG1 function result in a further increase in glycolate accumulation. The accumulation of photorespiratory metabolites when exposed to low CO₂ conditions is consistent with the slow growth phenotype and with a role of BASS6 in photorespiratory metabolism.

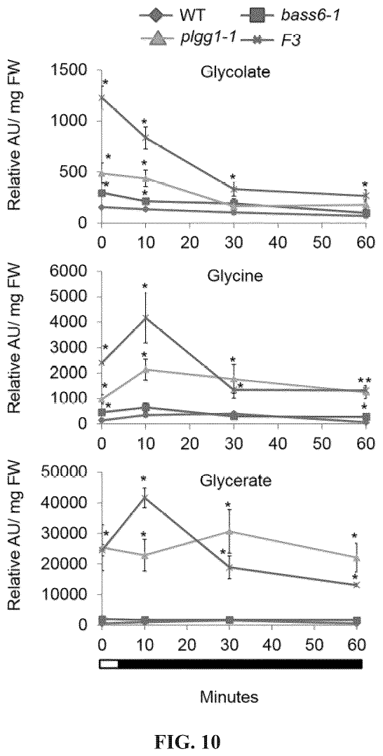

Photorespiration is a light dependent pathway. Metabolite analysis of the plgg1-1 Arabidopsis line showed a light dependent accumulation of glycolate, glycine, serine and glycerate (Pick et al., supra), consistent with impairment of both glycolate export and glycerate import in the chloroplast. During the night under ambient air, the glycolate and glycine levels in plgg1-1 mutants return to wild type levels and there was a significant reduction in glycerate (Pick et al., supra). To determine changes in glycolate, glycerate and glycine levels in bass6-1 and the F3 double mutant plants compared to wild type and plgg1-1, metabolite analysis was performed after shift from high [CO₂] to ambient air and immediately after the light to dark transition at the end of the growth photoperiod. The plgg1-1 mutants show a reduction in glycolate levels after the end of the light period (FIG. 10). The metabolite profile of bass6-1 plants showed accumulation of glycolate and glycerate at the end of the light period, which were significantly reduced within 10 minutes (FIG. 10). Accumulation of glycolate was significantly increased in the F3-bass6, plgg1 double mutant compared to plgg1-1 plants while glycine and glycerate levels in the F3 mutants were very similar to plgg1 mutants. These combined with the localization of BASS6 to the chloroplast inner envelope membrane strongly implies that PLGG1 and BASS6 are together responsible for the export of glycolate from chloroplasts.

### Example 5: BASS6 and PLGG1 Rescue Growth of Yeast on Glycolate as a Carbon Source

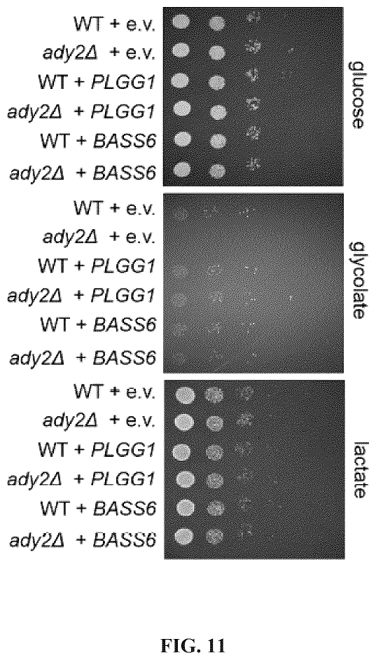

There is no known glycolate transporter in the yeast Saccharomyces cerevisiae, but the acetate transporter ADY2 is homologous to Escherichia coli yjcG that is known to transport both acetate and glycolate (Gimenez et al., J. Bacteriol., (2003) 185:6448-55). Therefore, we generated yeast vectors expressing both BASS6 and PLGG1 and expressed them in both wild type BY4741 and the isogenic ady2Δ strain. Spot assays to measure growth demonstrated that the ady2Δ strain expressing only empty vector was unable to grow on glycolate as the sole carbon source (FIG. 11). As predicted, the expression of PLGG1 in the ady2Δ yeast strain rescued growth back to the levels of wild type using glycolate as the sole carbon source (FIG. 11). Expression of the Bass6 gene also rescued growth in the ady2Δ strain, evidence that BASS6 can also complement for glycolate transport (FIG. 11). Controls using glucose and lactate as a carbon source show that the expression of BASS6 and PLGG1 in yeast does not negatively affect growth and that the ady2Δ strain can utilize both sources of carbon (FIG. 11).

Based on the findings that BASS6 and PLGG1 can complement yeast for growth on glycolate as a carbon source we sought to determine the transport capabilities of both proteins. To test transport characteristics of BASS6 and PLGG1 expressed in yeast we performed uptake experiments using [¹⁴C]-glycolic acid. [¹⁴C]-glycolic acid was incubated for 10 minutes before quenching and scintillation counting. The expression of PLGG1 protein showed an increase in the capacity for uptake of glycolate in both wild type and the ady2Δ strain (FIG. 12) as did the expression of BASS6 protein (FIG. 12). The data indicate that both BASS6 and PLGG1 expressed in yeast facilitate transmembrane glycolate transport leading to rescue of the growth in glycolate uptake defective ady2Δ mutant (FIG. 12).

### Example 6: Effects on Gene Expression

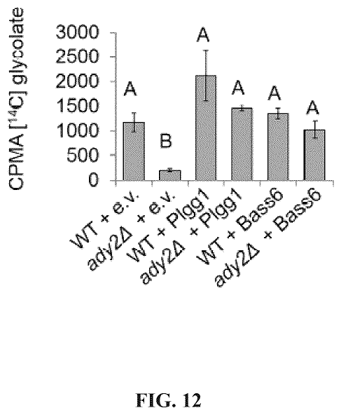

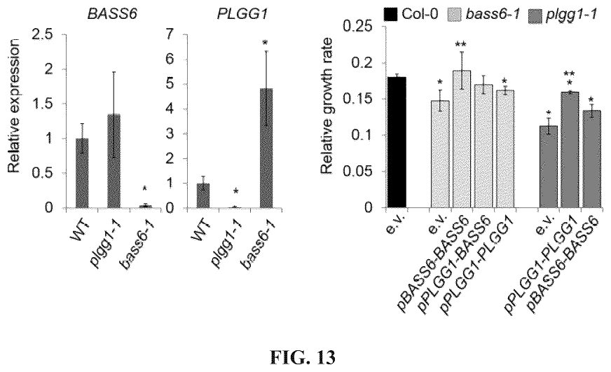

The loss of either Bass6 or Plgg1 in Arabidopsis leads to a photorespiratory mutant phenotype with reduced growth rates compared to wild type in ambient or lower [CO₂] air (FIG. 2). In addition, double mutant plants lacking the expression of BASS6 and PLGG1 further reduced the plant's ability to grow (FIG. 6A). Although the loss of either BASS6 or PLGG1 resulted in a photorespiratory phenotype, neither T-DNA insertion line was lethal when grown in ambient air as seen with numerous other photorespiratory mutants. This could be due to redundancy in the transport processes and compensation for loss of one gene by the increase in expression of another. To test if the loss of Bass6 or Plgg1 results in changes in expression of the other gene, Real-Time PCR (RT-PCR) experiments were performed. In the plgg1-1 line when compared to wild type there was no detectable difference in Bass6 expression (FIG. 13). However, the expression of Plgg1 was increased 4.8 fold over wild type in the bass6-1 line suggesting that Plgg1 expression markedly increased to compensate for metabolic changes caused by the loss of BASS6 (FIG. 13). After determining that Plgg1 expression increased in the bass6-1 plants, it was hypothesized that the change in Plgg1 expression is the reason there is a less severe phenotype observed in bass6-1 compared to plgg1-1. This led to testing whether the expression of either Bass6 or Plgg1 could potentially complement the slow growth phenotype of each of the single mutants.

Also, to rule out the possibility that either the plgg1-1 or the bass6-1 line phenotypes were due to another mutation, Plgg1 and Bass6 were also transformed into the plgg1-1 and the bass6-1 lines under the control of their native promoters. Expression of Bass6 under the control of its own promoter or the control of the Plgg1 promoter rescues the growth rate phenotype in the bass6-1 line, confirming the loss of BASS6 is the cause of the photorespiratory phenotype (FIG. 13). In addition, expression of Plgg1 under the control of its own promoter, rescues its photorespiratory phenotype (FIG. 13). However, transforming the expression plasmid of Plgg1 into the bass6-1 line showed no significant change in growth rate compared to the bass6-1 mutant (FIG. 13). This could be due to the fact that too much expression of PLGG1 can have a negative impact on plant growth and that the expression of the endogenous Plgg1 gene is already increased compared to wild type (Yang et al., supra; FIG. 13). Intriguingly, expression of Bass6 under the control of its native promoter somewhat increased the growth rate of the plgg1-1 line compared to the empty vector but was not completely rescued back wild type level (FIG. 13).

### Example 7: Enhancing Glycolate Flux Through Synthetic Bypass Pathways to Increase Plant Growth and Yield

### Plant Material

Nicotiana tabacum c.v. “Petite Havana” was transformed using Agrobacterium tumefaciens mediated transformation using standard methodology (Glowacka et al, Plant Cell Environ., (2016) 39:908-17) with 18 binary plasmids were assembled as described and listed in Table 5. The following abbreviations are utilized in the table: (TSR) tartonic semialdehyde reductase, (Spm) Maize Supressor-mutator transposable element promoter, (RbcS) Rubisco small subunit promoter and signal peptide, (Ocs) Agrobacterium opine synthase, (GdD) E. coli glycolate dehydrogenase subunit D, (Act2) Actin 2 promoter and terminator, (35s) Cauliflower mosaic virus 35s promoter and terminator, (Pgm) Phosphoglucomutase signal peptide, (GdE) E. coli glycolate dehydrogenase subunitE, (GdF) E. coli glycolate dehydrogenase subunit F, (Gel) glyoxylate carboligase, (GO) glycolate oxidase, (MS) malate synthase, (Cat) Catalase, (Nos) Agrobacterium Nopine synthase promoter and terminator, (2×35S) double 35s promoter, (Ubi) Ubiquitin promoter.

Bypass pathway 1 genes originated from E. coli and bypass pathway 2 genes originated from plant and E. coli sources as reported previously (Kebeish et al, Nat. Biotechnol. (2007) 25:593-99; Maier et al, Front. Plant Sci., (2012)). We developed a different pathway, Bypass pathway 3, utilizing genes originating from Chlamydomonas reinhardtii for glycolate dehydrogenase (SEQ ID NO: 44) and a gene originating from Cucurbita maxima for malate synthase (SEQ ID NO: 42). Along with these genes, we developed an RNAi module that targets the plastidic glycolate/glycerate transporter PLGG1 from A. thaliana that was designed using 300 bp of exon sequence (SEQ ID NO: 46) derived from the Sol genomics network (solgenomics.net). All binary plasmids contained the BASTA resistance (bar) gene as a selectable marker for plant transformation. A minimum of 10 independent To transformations were generated to produce Ti progeny. T-DNA copy number was determined on Ti either by digital droplet PCR analysis or through qRT-PC analysis (iDNA genetics, Norwich UK). From these results a minimum of 5 independent transformation events were selected to self and produce T₂ progeny. Copy number analysis was performed again to verify single insert homozygous lines for each transformation event.

TABLE 5
Synthetic glycolate utilization pathways

| | | | | |
|---|---|---|---|---|
| Plasmid | Inserted gene | Promoter | Signal peptide | Terminator |

Bypass 1

| | | | | |
|---|---|---|---|---|
| EC27180 | TSR | Spm | RbcS | Ocs |
| | GdD | RbcS | Pgm | Mas |
| | GdE | Act2 | RbcS | Act2 |
| | GdF | 35 s | Pgm | Act2 |
| | Gcl | 2 × 35 s | Pgm | 35 s |
| EC27181 | Gcl | Spm | RbcS | Ocs |
| | TSR | RbcS | Pgm | Mas |
| | GdD | Act2 | RbcS | Act2 |
| | GdE | 35 s | Pgm | Act2 |
| | GdF | 2 × 35 s | Pgm | 35 s |
| EC27182 | GdF | Spm | RbcS | Ocs |
| | Gcl | RbcS | Pgm | Mas |
| | TSR | Act2 | RbcS | Act2 |
| | GdD | 35 s | Pgm | Act2 |
| | GdE | 2 × 35 s | Pgm | 35 s |
| EC27183 | GdE | Spm | RbcS | Ocs |
| | GdF | RbcS | Pgm | Mas |
| | Gcl | Act2 | RbcS | Act2 |
| | TSR | 35 s | Pgm | Act2 |
| | GdD | 2 × 35 s | Pgm | 35 s |
| EC27184 | GdD | Spm | RbcS | Ocs |
| | GdE | RbcS | Pgm | Mas |
| | GdF | Act2 | RbcS | Act2 |
| | Gcl | 35 s | Pgm | Act2 |
| | TSR | 2 × 35 s | Pgm | 35 s |
| EC27186 | TSR | Spm | RbcS | Ocs |
| | GdD | RbcS | Pgm | Mas |
| | GdE | Act2 | RbcS | Act2 |
| | GdF | 35 s | Pgm | Act2 |
| | Gcl | 2 × 35 s | Pgm | 35 s |
| | PLGG1 RNAi | Ubi | | |
| EC27187 | Gcl | Spm | RbcS | Ocs |
| | TSR | RbcS | Pgm | Mas |
| | GdD | Act2 | RbcS | Act2 |
| | GdE | 35 s | Pgm | Act2 |
| | GdF | 2 × 35 s | Pgm | 35 s |
| | PLGG1 RNAi | Ubi | | |
| EC27188 | GdF | Spm | RbcS | Ocs |
| | Gcl | RbcS | Pgm | Mas |
| | TSR | Act2 | RbcS | Act2 |
| | GdD | 35 s | Pgm | Act2 |
| | GdE | 2 × 35 s | Pgm | 35 s |
| | PLGG1 RNAi | Ubi | | |
| EC27189 | GdE | Spm | RbcS | Ocs |
| | GdF | RbcS | Pgm | Mas |
| | Gcl | Act2 | RbcS | Act2 |
| | TSR | 35 s | Pgm | Act2 |
| | GdD | 2 × 35 s | Pgm | 35 s |
| | PLGG1 RNAi | Ubi | | |
| EC27194 | GdD | Spm | RbcS | Ocs |
| | GdE | RbcS | Pgm | Mas |
| | GdF | Act2 | RbcS | Act2 |
| | Gcl | 35 s | Pgm | Act2 |
| | TSR | 2 × 35 s | Pgm | 35 s |
| | PLGG1 RNAi | Ubi | | |

Bypass 2

| | | | | |
|---|---|---|---|---|
| EC27171 | GO | Nos | pgm | Nos |
| | MS | Spm | RbcS | Ocs |
| | CAT | 2 × 35 s | pgm | 35 s |
| EC27172 | CAT | Nos | pgm | Nos |
| | GO | Spm | RbcS | Ocs |
| | CmMS | 2 × 35 s | pgm | 35 s |
| EC27173 | CmMS | Nos | pgm | Nos |
| | CAT | Spm | RbcS | Ocs |
| | GO | 2 × 35 s | pgm | 35 s |
| EC27174 | GO | Nos | pgm | Nos |
| | CmMS | Spm | RbcS | Ocs |
| | CAT | 2 × 35 s | pgm | 35 s |
| | PLGG1 RNAi | | | |
| EC27175 | CAT | Nos | pgm | Nos |
| | GO | Spm | RbcS | Ocs |
| | CmMS | 2 × 35 s | pgm | 35 s |
| | PLGG1 RNAi | | | |
| EC27176 | CmMS | Nos | pgm | Nos |
| | CAT | Spm | RbcS | Ocs |
| | GO | 2 × 35 s | pgm | 35 s |
| | PLGG1 RNAi | Ubi | | |

Bypass 3

| | | | | |
|---|---|---|---|---|
| EC27200 | CrGDH | Act2 | RbcS | Act2 |
| | CmMS | Spm | RbcS | Ocs |
| EC27201 | CrGDH | Act2 | RbcS | Act2 |
| | CmMS | Spm | RbcS | Ocs |
| | PLGG1 RNAi | Ubi | | |

### Chlorophyll Fluorescence Measurements

Tobacco seeds were germinated under ambient air conditions on Murashige and Skoog (MS) plates with essential vitamins in a controlled environment chamber (Environmental Growth Chambers, Chagrin Falls, Ohio, USA) with 14 h day (25° C.)/10 h night (22° C.) and light intensity of 500 μmol m⁻² s⁻¹. Eight days after germination, seedling plates were transferred to a custom assembled low CO₂ chamber inside the controlled environment growth chamber. The light levels were increased to 1200 μmol m⁻² s⁻¹ for 24 hours and CO₂ concentration was maintained below 35 pbar. Fv′/Fm′ was determined on each plate using the CF Imager Technologica (www.technologica.co.uk). Maximum flash intensity was 6800 μmol·m⁻² s⁻¹ for 800 milliseconds. Image values were obtained for each individual plant by detecting colonies within the fluorimager software program defining each position as has been previously described (South et al, Plant Cell (2017) 29:808-823; Badger et al, Funct. Plant Biol. (2009) 36:867-73; Schmidt & Delaney, Mol. Genet. Genomics (2010) 283:233-41).

### Gene Expression and Protein Detection

Plants were grown under greenhouse or field conditions described below. Five leaf discs were harvested from three plants per line (2.9 cm², ˜100 mg). RNA and protein were extracted from the same leaf samples using the NucleoSpin RNA/Protein kit (Macherey-Nagel GmbH & Co.KG, Duren, Germany). cDNA was generated from extracted RNA using the Quantinova reverse transcriptase kit (QIAGEN, USA). A minimum of three biological replicates including three technical replicates each were used for all samples. Gene expression was analyzed using a Bio-Rad CFX connect real-time PCR system (Bio-Rad Laboratories, USA). Relative changes in transcript were determined using the ΔΔCt method using primers directed toward the transgene transcripts and the L25 gene as a standard control gene (Brooks & Farquhar, Planta (1985) 165:397-406. cDNA was amplified using a SSO advanced SYBR green master mix (Bio-Rad) and primer sequences are described in Table 6.

TABLE 6
Primers for gene expression analysis

| Primer Name | SEQ ID NO: | Sequence |
|---|---|---|
| L25 RT F | SEQ lD NO: 22 | CCCCTCACCACAGAGTCTGC |
| L25 RT R | SEQ lD NO: 23 | AAGGGTGTTGTTGTCCTCAATCTT |
| PLGG1 Nt | SEQ lD NO: 24 | CTCAAATAAAGTTGAAATCCTTAC |
| RT-1F | | AAAC |
| PLGG1 Nt | SEQ lD NO: 25 | TCTTGGTAGGGATGAATTGGAC |
| RT-2R | | |
| RT-MS-001F | SEQ lD NO: 26 | GGGAATCTGAGTGGACATGTG |
| RT-MS-002R | SEQ lD NO: 27 | CCAGAATTGAGTGCGTTGATG |
| RT-GO-001F | SEQ lD NO: 28 | ACAGAAACGCTTTTGCAAGG |
| RT-GO-002R | SEQ lD NO: 29 | GGTGAGCCATCTTTTGCATG |
| RT-CAT-001F | SEQ lD NO: 30 | GCGAGAAAATCACCCACTTTG |
| RT-CAT-002R | SEQ lD NO: 31 | TGGCTGGAAATAACCGTGAG |
| RT-TSR-001F | SEQ lD NO: 32 | TGAATTACTGTCGCTGGGC |
| RT-TSR-002R | SEQ lD NO: 33 | GTACAACCATTTTCACCGAACAG |
| RT-GCL-001F | SEQ lD NO: 34 | ATCAATCCGTTCTACTCAGCG |
| RT-GCL-002R | SEQ ID NO: 35 | GACATACGCCGATATTCCCTG |
| RT-GdD-001F | SEQ ID NO: 36 | GGAGGTAGCATCTTGTACGAAG |
| RT-GdD-002R | SEQ ID NO: 37 | CGGTATGCAGGATCTCAAGTC |
| RT-GdE-001F | SEQ ID NO: 38 | CGAGTGTGATTACAGCCAGG |
| RT-GdE-002R | SEQ ID NO: 39 | TGACAACGAACATCCAGCG |
| RT-GdF-001F | SEQ ID NO: 40 | CTGTGTTCACTGCGGATTTTG |
| RT-GdF-002R | SEQ ID NO: 41 | CTCCTGTGTTTTAAGCGTGAC |

Total protein from Bypass 3 was extracted using the Nucleospin protein/RNA kit above, or from frozen leaf material ground in liquid nitrogen, resuspended in lysis buffer (50 mM Hepes pH 7.6, 300 mM sucrose, 2 mM MgCl₂) plus plant protease inhibitor cocktail (Sigma-Aldrich). Protein was quantified using the protein quantification assay (Macherey-Nagel GmbH & Co.KG, Düren, Germany). 3 μg of protein was loaded per lane and separated by SDS-polyacrylamide electrophoresis (SDS-PAGE). PAGE gels were transferred to PVDF membranes (Immobilon-P, Millipore, USA) using a Bio-Rad semi-dry transfer system. After blocking in a 6% milk TBS-T solution, membranes were incubated with custom antibodies raised against the malate synthase (MS) and PLGG1 (Agrisera, Vannas, Sweden) and glycolate dehydrogenase (GDH) (Genscript, USA). As a protein loading control a commercial antibody raised against the large subunit of Rubisco (RbcL) was used (Agrisera, Vannas, Sweden). After subsequent washing and incubation with anti-rabbit secondary antibody (Bio-Rad, USA) Chemiluminescence was detected using the ImageQuant LAS4010 scanner (GE Healthcare Life Sciences, Pittsburgh, USA).

### Growth Analysis (Greenhouse)

To determine if the three bypasses to photorespiration would result in increased growth capacity and growth rate, stem height, and dry weight biomass was determined. Single insert T₂ seeds were germinated on LC1 sunshine mix (Sun Gro 202 Horticulture, Agawam, MA, USA). 10 days after germination seedlings were transferred to 4 L pots (400 C, Hummert International, Earth City, MO, USA) with LC1 sunshine mix supplemented with slow release fertilizer ((Osmocote Plus 15/9/12, The Scotts Company LLC, Marysville, OH, USA). Pots were randomized within the greenhouse and positions were changed before each watering. Light intensity within the greenhouse was measured using a quantum sensor (LI-190R, LI-COR, Lincoln, Nebraska, USA). Air temperature, relative humidity and [CO2] were measured using a combined temperature and humidity sensor (HMP60-L, Vaisala Oyj, Helsinki, Finland) and an infrared gas analyzer (SBA-5, PPsystems, Amesbury, MA, USA). All climate data was logged using a data logger (CR1000, Campbell Scientific Inc, Logan, UT, USA). Greenhouse growth conditions utilized were similar to those previously reported in the literature (Kromdijk et al, supra). Above ground biomass was harvested at seven weeks after determination of stem height and dried for 2 weeks and dry weight was determined for each fraction.

### 2016 Field Experiment

As a proof of concept experiment, the effect of each photorespiration bypass design was evaluated under field conditions for the 2016 season in central Illinois. Five independent transformation events of Bypass 3 four events of Bypass 1 and due to poor performance compared to WT, only two independent transformations of bypass 2, with two wild type (WT) and two empty vector (EV) controls were planted in a randomized block design. Homozygous single insert T₂ seeds were germinated in pots containing soil mix (Sun Gro 202 Horticulture, Agawam, MA, USA) on May 14, 2016 and grown for seven days then transferred to floating trays as previously described (Kromdijk et al, Science (2016) 354:857-61). Plants were transplanted to the Universtiy of Illinois Energy Farm field station (40.11°N, 88.21 261° W, Urbana, IL, USA) on Jun. 6, 2016 after the field was prepared as described (Kromdijk et al, supra). Each block was 6×6 spaced 30 cm apart. The internal 16 plants per block were the indicated transgenic plant lines surrounded by a WT border. An additional two row border of WT plants surrounded the experiment. Watering was provided as needed from six water towers placed within the plot. Weather data, including Light intensity, air temperature, and precipitation were measured for the 2016 field season as described (data not shown).

Apparent quantum efficiency of photosynthesis (Φa) including the light saturated level of photosynthesis at ambient 400 μbar and low 100 μbar CO₂ concentrations was measured on the youngest fully expanded leaf 14-20 days after transplanting to the field. (Pa was determined from assimilation measurements in response to light levels at the indicated [CO₂]. Gas exchange measurements were performed using a LI-COR 6400XT with a 2 cm² fluorescence measuring cuvette with gasket leaks corrected for as outlined in the manual (LI-COR Biosciences, Lincoln, NE, USA). Measurements of CO₂ assimilation were done at light intensities of 1200, 380, 120, 65, 40, 30, 25, 18, and 10 μmol·m⁻²·s⁻¹, assimilation was recorded after a minimum of 120 seconds at each light level. (Pa was calculated from the slope of the initial response of assimilation at low light levels. The saturating level of assimilation (Aₛₐₜ) was determined from the 1200 μmol·m⁻²·s⁻¹ measurement at the indicated [CO₂]. Stem height, leaf and stem biomass was determined for 8 plants per plot at 7 weeks post planting. After stem height was assessed, above ground biomass was harvested and separated into leaf and stem fractions. Plant material was dried for a minimum of 2 weeks prior to biomass measurements.

### 2017 Field Experiment

To get a more accurate evaluation of the effect of Bypass 3 on plant productivity under agricultural conditions a repeated randomized block design was used for the 2017 field season. The field plot consisted of five replicate blocks with seven randomized 6×6 plots per block. The central 16 plants were the tested transgenic lines, or WT surrounded by a WT border. The entire 35 plots were surrounded by additional rows of WT as a border. Single insert homozygous lines from the same harvest were sown on LC1 sunshine mix and germinated for seven days. After seven days, seedlings were transplanted to floating trays as described above. 14 days after transplant to floating trays, plants were transplanted to the Energy farm field station at the University of Illinois, Urbana, IL USA on Jun. 21, 2017. Watering was provided as needed using parallel drip irrigation (drip line). Weather data, including Light intensity, air temperature, and precipitation were recorded for the 2017 field season. Photosynthesis measurements to determine the (Pa were performed Jul. 2-5, 2017 along with photosynthetic pigments harvested during the same time on the youngest fully expanded leaf.

Φa was performed as previously described (Kromdijk et al., supra). Briefly, gas exchange measurements were performed using a LI-COR 6400XT with a 2 cm² fluorescence measuring cuvette as described above. Measurements of CO₂ assimilation response to light were started pre-dawn and were performed at light intensities of 0, 10, 18, 25, 30, 40, 65, 120, 380, 1200, and 2000 μmol mol⁻¹. Diurnal measurements of photosynthesis were performed starting pre-dawn on July 14 and measured every two hours on two plants per block. Light levels and temperature were determined prior to measurements based on incoming light levels using a PAR sensor on the LI-COR 6400 and built in temperature sensor. CO₂ concentration was maintained at 400 ppm. Diurnal measurements were continued until after dusk. At 49 days post germination, eight plants per plot were harvested from all five replicate blocks. Above ground biomass was separated into leaf and stem fractions and dried for 2 weeks before biomass measurements. For starch analysis, 10 mg of leaf material collected on July 14^{th} was frozen in liquid nitrogen and stored at −80° C. until processing. Starch was assayed using the Enzychrom starch assay kit (bioassay systems, Hayward, CA, USA). Colorimetric measurements were performed on a Biotek synergy HT plate reader (Biotek Winooski, VT, USA).

### Gas Exchange

To determine the net photosynthetic assimilation rate from a CO₂ dose response the fifth leaf from the base of seven-week-old N. tabacum plants were clamped into the fluorescence cuvette of a LI-COR 6800 infrared gas analyzer (Li-Cor Biosciences, Lincoln, NE, USA) with leaf temperature controlled at 25° C. and light intensity set at 1500 μmol m⁻² s⁻¹. Leaves were acclimated at 400 μmol mol¹ to achieve a steady state. The CO₂ concentration of the response curve was set at 400, 200, 100, 50, 30, 400, 600, 800, 1000, 1500, 2000 μmol mol¹ and measurements were taken when assimilation reached a steady state. To determine the maximum rate of carboxylation (V_{cmax}), maximum electron transport rate (Jₘₐₓ) and mitochondrial respiration rate a model for leaf photosynthesis with temperature corrections was used assuming infinite mesophyll conductance from the collected CO₂ response curves. F and R_{d} measurements using the common intersection method Gas exchange was performed using a LI-COR 6800 (LI-COR Biosciences) using a fluorescence chamber. Γ* was measured using the common intersection method by measuring the CO₂ response of photosynthesis under various sub-saturating irradiances. The common intersection was determined using slope-intercept regression to produce more accurate and consistent values of Ci* and R_{d} (Walker et al, Plant Cell Environ. (2016) 39:1198-1203). Plants were acclimated under 250 μmol m⁻² s⁻¹ light at 150 μBar CO₂ until photosynthesis reached steady and measured at 150, 120, 90, 70, 50, and 30 μBar CO₂ under irradiances of 250, 165, 120, 80, and 50 μmol m⁻² s⁻¹. The x-intersection point was converted to Γ* as previously reported (Walker et al, supra).

### Statistical Analysis

All statistical analysis was performed using Origin pro 2016 (version 9.3.226, Origin lab corporation Northampton, MA, USA). For Fv′/Fm′ measurements, each plate contained a minimum of 10 seedlings and data indicates average values. Significance was evaluated by one-way analysis of variance (ANOVA). Relative changes in gene expression were analyzed by one-way ANOVA with three technical replicates per biological replicate from either greenhouse or field grown samples. Greenhouse biomass and stem height experiments were analyzed by a one-way ANOVA with a minimum of 8 biological replicates. Biomass and Stem height experiments from the 2016 field season were analyzed by a one-way ANVOA with 8 biological replicates. Biomass data from the 2017 field season was analyzed by a two-way ANOVA (genotype×block) with 8 biological replicates per genotype per block. Greenhouse photosynthetic measurements were analyzed by a one-way ANOVA and three biological replicates per measurement and field photosynthetic measurements were analyzed by a two-way ANOVA with two plant replicates per plot and five randomized replicate blocks. All ANOVA testing was performed with a P<0.05 or smaller as indicated in figures. All ANOVA analysis was followed with a Tukey's post-hoc test for means comparison.

### Results and Analysis

Nicotiana tabacum was transformed with three different photorespiration bypass designs expressing as many as 5 genes (FIG. 14, Table 5). Bypass 1 and Bypass 2 were previously reported in the literature. However, the newly developed Bypass 3 was designed utilizing the Chlamydomonas reinhardtii glycolate dehydrogenase (SEQ ID NO: 45) instead of glycolate oxidase which beneficially does not produce hydrogen peroxide as a byproduct during the conversion of glycolate to glyoxylate (Abolemy et al, Plant Physiol. Biochem. (2014) 79:25-30).

Unlike the testing of single gene inserts, multigene constructs may need increased coordination of gene expression to optimize flux through the designed pathway. Without a priori knowledge of the promoter gene combinations that optimize efficacy of photorespiration bypass, we utilized multiple promoter gene combinations for the reported photorespiratory bypass designs, five iterations of bypass 1, three iterations of Bypass 2 and a single iteration of Bypass 3 were generated (Table 5). In addition to the expression of the photorespiratory bypass genes, a long hairpin RNAi construct (SEQ ID NO: 46) was designed and added to the library of multigene constructs to reduce the expression of the chloroplast glycolate/glycerate transporter PLGG1 with the goal of increasing flux through the bypass pathways (FIG. 14, Table 5). In total, 17 of 18 independent constructs designed were successfully transformed and examined to test the function of Bypass 1, 2, and 3, with and without the inclusion of an RNAi module targeting the PLGG1 transporter.

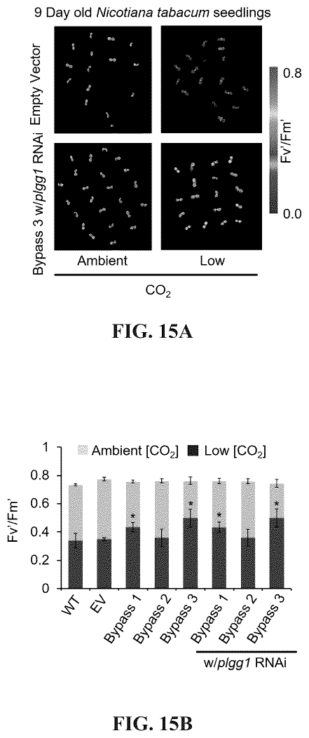

Photorespiratory stress induced damage to photosystem II can be visualized with chlorophyll fluorescence via decreases in maximal operating efficiency of PSII in the light (i.e. Fv′/Fm′) (South et al., supra; Badger et al., supra). Using these changes in fluorescence as an indication of apparent photorespiration efficiency, each photorespiratory bypass design was screened after 24 hours of growth at high light (1200 mol m⁻² s⁻¹) and near zero concentrations of CO₂, and compared to wild-type (WT) and empty vector (EV) controls (FIGS. 15A and 15B). Overall, plants transformed with versions of Bypass 1 and Bypass 3 showed improved apparent photorespiration efficiency compared to WT and EV controls (FIG. 15B). From this initial screen, lines demonstrating enhanced apparent photorespiration efficiency were selected from each design for further characterization in both greenhouse and field settings.

During and after initial assessment of the multiple gene-construct designs, many prototypes demonstrated poor phenotypes, either due to independent insertion, or sub-optimally designed promoter gene combinations, with multiple insertion events having the same detrimental phenotype.

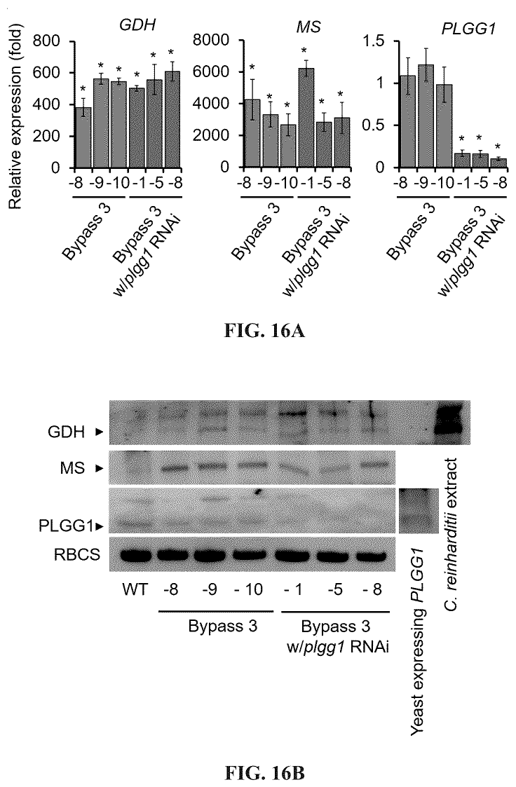

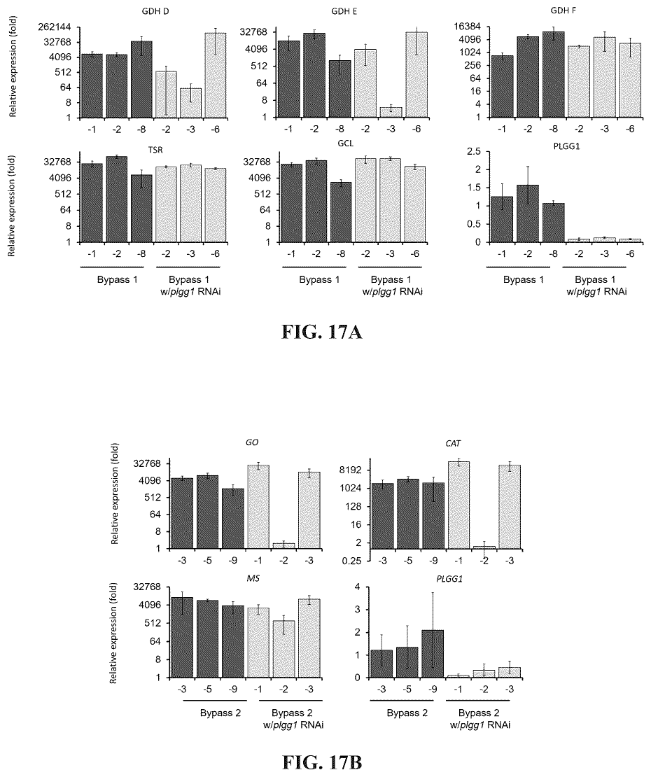

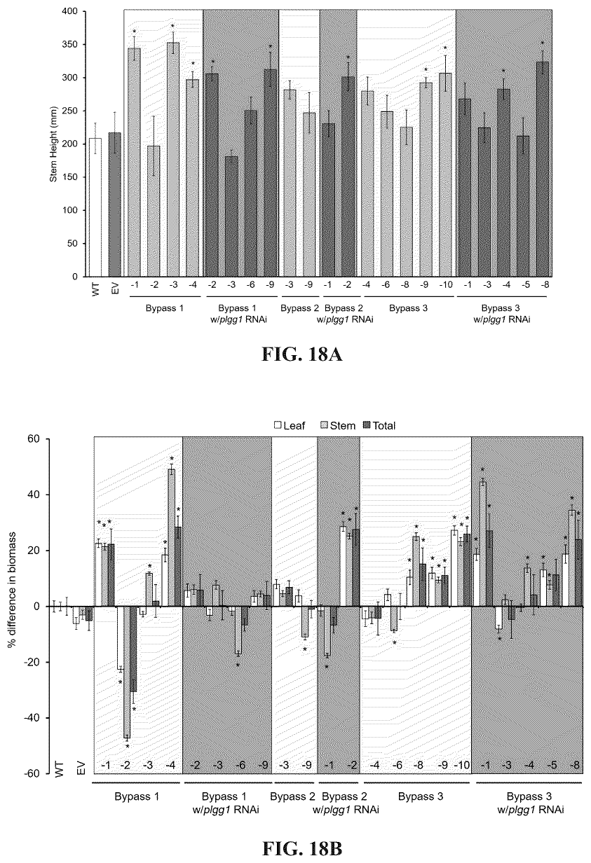

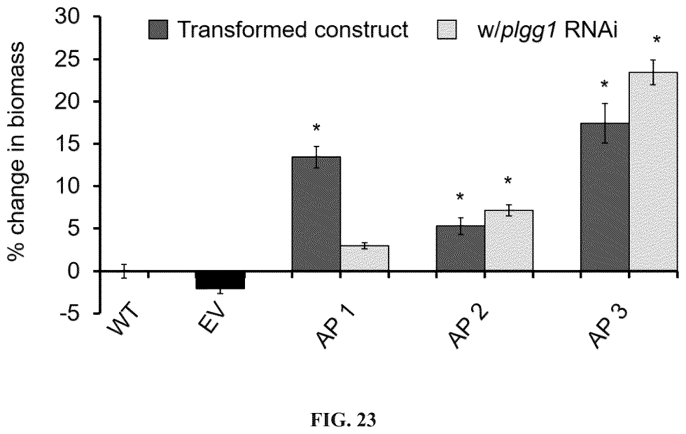

After successful screening, gene expression of the photorespiration bypass pathways was verified for each construct further tested in greenhouse and field trials (FIG. 16A and FIGS. 17A and 17B). A minimum of three independent transformations of each construct design were assessed under greenhouse conditions. We observed increases in dry weight biomass in all three bypass designs, suggesting a successful photorespiration bypass similar to previously reported findings in other plant species (Dalal e al., Biotechnol Biofuels (2015) 8; Kebeish et al., supra; Maier et al., supra; Nolke et al., Plant Biotechnol. J. (2014) 12:734-42; Ahmad et al Plant Biotechnol. Rep. (2016) 10:269-76). Overall, under greenhouse conditions, plants from the novel Bypass 3 exhibited unexpectedly greater differences in biomass than plants from Bypass 1 and 2 lines, and we observed a further enhancement in Bypass 3 when the RNAi module targeting PLGG1 was present, with total biomass increasing by as much as 23% relative to WT and 13% and 7% compared to Bypass 1 and 2 lines, respectively (FIG. 23). When tested under field conditions Bypass 3 with the RNAi targeting PLGG1 again showed the most significant increases in total dry weight biomass by as much as 27% as compared to the WT control (FIG. 18B).

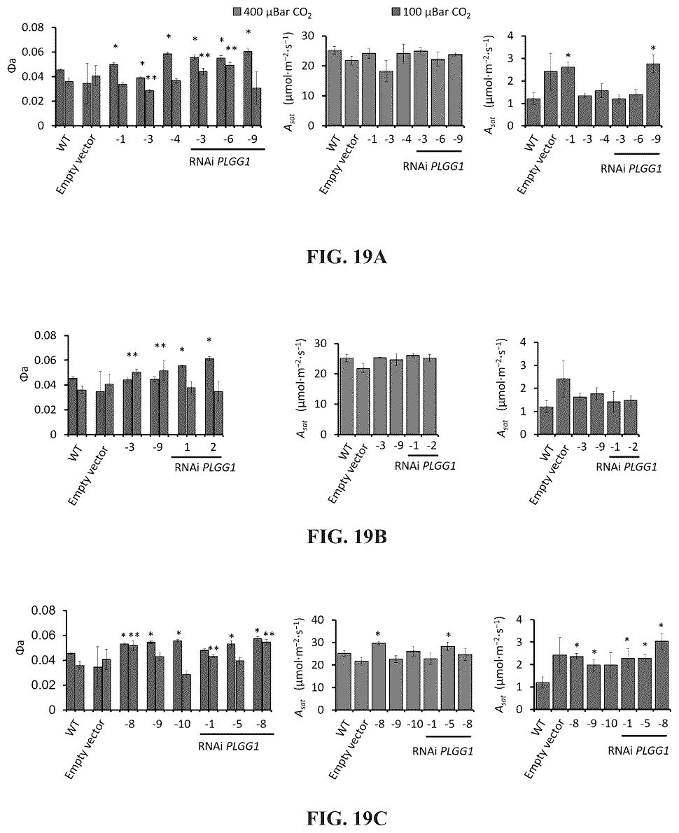

Promising lines from greenhouse trials were then tested for increased photosynthetic efficiency and plant productivity under agricultural conditions in a single block replicated garden plot experiment in 2016. We hypothesized that plants with bypass designs would exhibit increases in the quantum efficiency of photosynthesis (Φa) due to their decreased metabolic flux through the native photorespiratory pathway. Overall, we observed increases in the (a in plants from all bypass lines, including those containing the RNAi module targeting the PLGG1 transporter (FIG. 19A-19C). We also measured performance under photorespiratory stress (i.e. low [CO₂]) conditions, and found that plants from Bypass 3 lines had an increased light-saturated rate and quantum efficiency of photosynthesis, again indicating a lessening of photorespiratory stress associated with a successful bypass design.

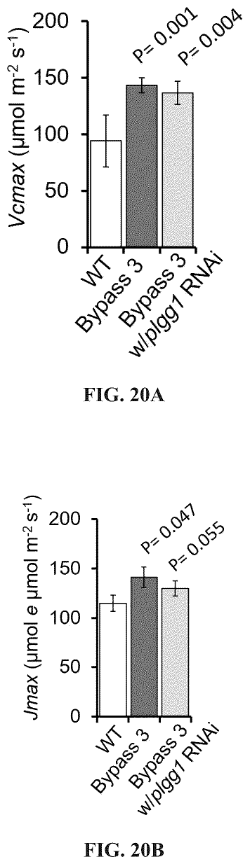

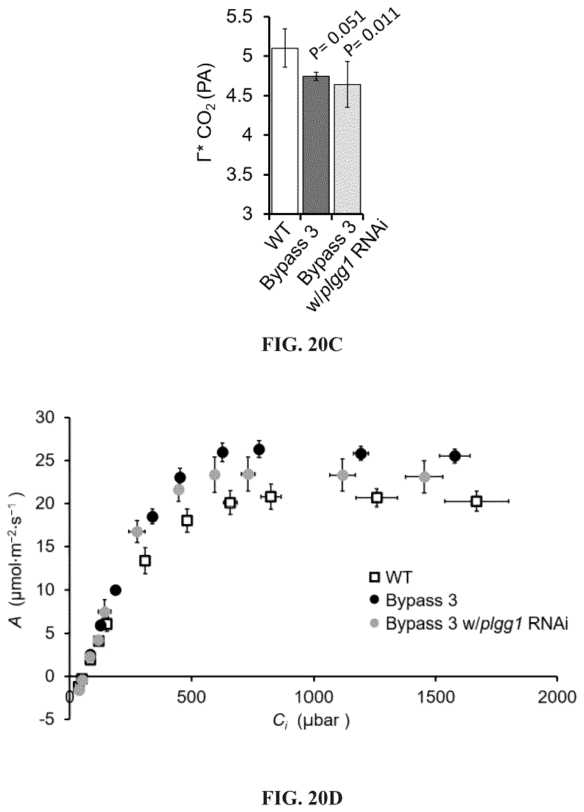

The combined fluorescence screen, greenhouse and 2016 field season studies show that bypass 3 was able to outperform WT, EV, and Bypass 1 and 2 in total plant growth, and this design was carried forward for further characterization. The Bypass 3 design was validated with Western blot analysis to ascertain the presence of CrGDH and MS as well as reduction in PLGG1 protein using custom generated antibodies (FIG. 16B). We further characterized the physiological impact of photorespiration Bypass 3 in planta under greenhouse conditions. We determined the maximum rate of carboxylation (V_{cmax}), and the RuBP limited rate of electron transport (Jₘₐₓ) by modelling photosynthetic rates (A) based on internal CO₂ concentration (Ci). Bypass 3, both with and without the PLGG1 RNAi module, demonstrated increases in V_{cmax} and in Jₘₐₓ suggesting more efficient photosynthesis at lower [CO₂] where photorespiration stress would be highest (FIG. 20A, 20B, 20D). We hypothesized that photorespiration bypass should lower the photosynthetic compensation point, or the point in which internal [CO₂] available for photosynthesis is equal to the CO₂ produced in daytime respiration. Indeed, we observed lower Γ* measurements in our bypass 3 plants compared to WT controls suggesting that photorespiration bypass increases photosynthetic efficiency at lower [CO₂] values possibly due to increases in the concentration of CO₂ within the chloroplast, which is predicted following the decarboxylation steps in the introduced pathway (FIG. 14).

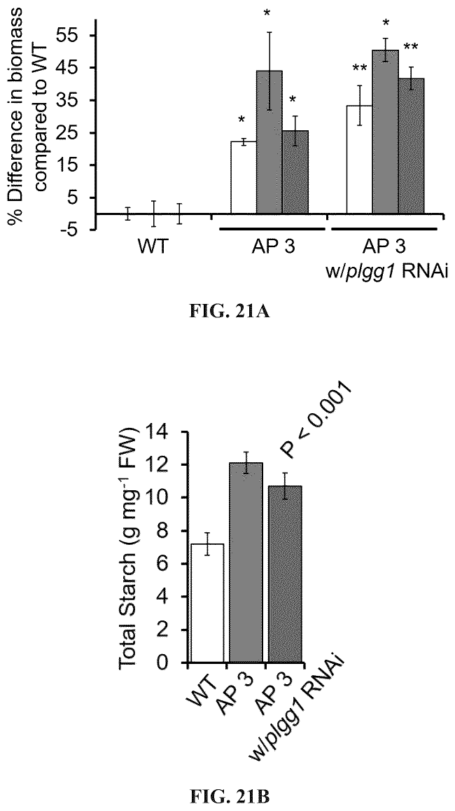

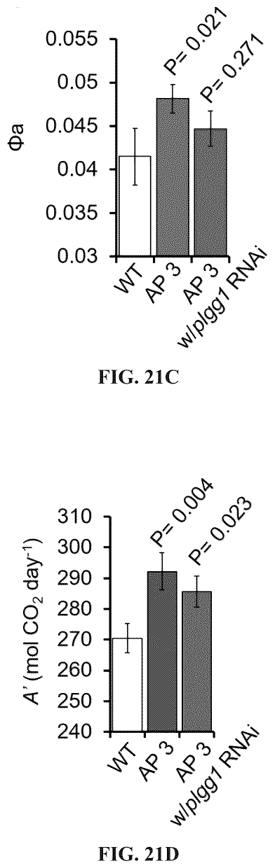

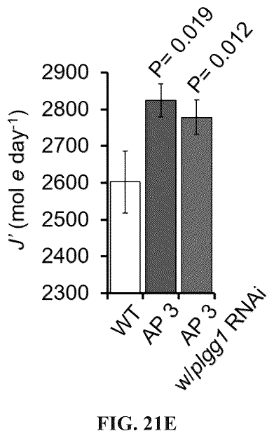

To better asses how Bypass 3 performs under agricultural conditions, a larger replicated block design was used during the 2017 field season. Five randomized replicate blocks were tested including three independent transformed bypass lines with and without the RNAi module targeting PLGG1. During the 2017 field season, we assessed leaf, stem, and total dry weight biomass, mid-day starch content, apparent quantum efficiency of photosynthesis ((Da). Overall, bypass 3 showed a 25% increase in total dry weight biomass (22% leaf, 44% stem) and Bypass 3 with PLGG1 RNAi showed a 41% increase in total dry weight biomass (33% leaf 50% stem) (FIG. 21A). In addition, the inclusion of the PLGG1 RNAi module in the Bypass 3 design showed a significant increase in leaf and total dry weight biomass compared to Bypass 3 alone (FIG. 21A). Total mid-day starch content was elevated in both Bypass 3 designs compared to the WT control by approximately 70% and 42% respectively (FIG. 21B). The apparent quantum efficiency of photosynthesis was increased in both bypass designs and significantly increased in bypass 3 alone (FIG. 21C).

With an increased quantum efficiency of photosynthesis and a decrease in the compensation point in both Bypass 3 designs, we hypothesized that the total net photosynthesis throughout the light period would be higher compared to the WT control resulting in the observed increases in biomass (FIG. 18B and FIG. 21A). To determine this, we measured the combined diurnal assimilation of CO₂, and observed significant increases in the total net assimilation in both bypass designs (A′) and the total number of electrons used toward photosynthesis (J′) compared to WT (FIG. 21D and FIG. 21E).

Overall, our synthetic biology approach let us design, build and test multiple photorespiration bypass designs and to compare different promoter gene combinations. In addition, this was the first study to describe the effects of photorespiration bypass under agriculturally relevant conditions, where the final results were not clearly predictable. The Bypass 1 design which was the first and currently the most reported design indeed shows improvements in plant growth and dry weight biomass (FIG. 18B). When compared, Bypass 1 was surprisingly less productive than Bypass 3 and the improvement of having Bypass 1 in place was reduced when the PLGG1 RNAi module was added in both greenhouse and field settings (FIGS. 18A and 18B). These data suggest that the Bypass 1 metabolic pathway cannot convert glycolate effectively when there's a reduction in flux through the native photorespiration pathway, i.e. when PLGG1 expression is targeted for silencing, or not expressed in a knockout strain. Bypass 2 showed the least improvements in plant productivity and many transgenic lines resulted in stunted growth and yellow leaves. The production of hydrogen peroxide as a byproduct and a non-optimized expression of catalase, which has been previously suggested, is likely the cause of the Bypass 2 phenotypes (Maier et al., supra).

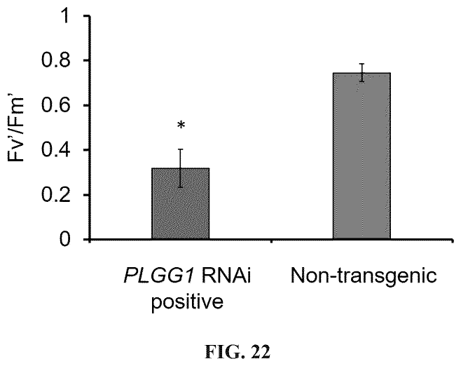

Photorespiration Bypass 3 which contains the C. maxima malate synthase and C. reinhardtii glycolate dehydrogenase enzyme, significantly increased plant biomass and demonstrated surprising improvements to photosynthetic efficiency over potential bypass pathways previously reported in the literature. In addition, the inclusion of an RNAi module that reduced the expression of the PLGG1 chloroplast glycolate glycerate transporter, resulting in an effect similar to the PLGG1 knockout strains (FIG. 22) significantly increased post-harvest dry weight biomass compared to Bypass 3 alone (FIG. 18B).

While the invention has been described with reference to details of the illustrated embodiments, these details are not intended to limit the scope of the invention as defined in the appended claims. The embodiment of the invention in which exclusive property or privilege is claimed is defined as follows:

What is claimed is:

1. A method of increasing quantum efficiency of photosynthesis and/or decreasing photorespiratory stress in a C3 plant, comprising introducing into a plant cell:
(a) a genetic alteration comprising a reduction or loss of expression or activity of an endogenous chloroplast inner membrane plastidic glycolate/glycerate translocator (PLGG1) protein in at least a portion of the chloroplasts of the C3 plant as compared to a control plant lacking the genetic alteration; and
(b) a first heterologous polynucleotide encoding a malate synthase and a second heterologous polynucleotide encoding a glycolate dehydrogenase, wherein the malate synthase and the glycolate dehydrogenase localize to a chloroplast of the plant,
wherein the quantum efficiency of photosynthesis is increased and/or the photorespiratory stress is decreased in the plant compared to a C3 plant not comprising (a) and (b).

2. The method of claim 1, wherein the PLGG1 protein is a protein having an amino acid sequence selected from the group consisting of SEQ ID NO: 6 (Arabidopsis thaliana NP_564388.1, AAM65181.1), Arabidopsis lyrata (XP_020868671.1, EFH69957.1), Arabis alpina (KFK44969.1), Capsella rubella (XP_006307262.1), Camelina sativa (XP_010478626.1, XP_010461027.1, XP_010499753.1), Citrus sinensis (XP_006471454.1), Brassica napus (XP_013733826.1, XP_013731498.1, XP_013731491.1, CDY59206.1, CDY22583.1, CDY35540.1, XP_022575243.1), Brassica rapa (XP_009114919.1, XP_009145211.1), Raphanus sativus (XP_018457661.1, XP_018486680.1), Brassica oleracea var. oleracea (XP_013587088.1, XP_013587305.1), Eutrema salsugineum (XP_006415255.1), Hevea brasiliensis (XP_021652349.1), Juglans regia (XP_018843901.1), Arabis alpina (KFK44969.1), Tarenaya hassleriana (XP_010518925.1), Ricinus communis (XP_002519004.1), a truncated, mutated, or otherwise modified version thereof, and an ortholog or functional homolog thereof from a C3 plant species;
wherein the protein performs the function of exporting glycolate from, and importing glycerate into, the chloroplast inner envelope membrane.

3. The method of claim 1, wherein the PLGG1 protein is an ortholog or functional homolog of the PLGG1 protein of Arabidopsis thaliana having the amino acid sequence set forth in SEQ ID NO: 6; or a sequence that is substantially similar to SEQ ID NO: 6 such that it performs the same function, hybridizes under similar conditions, or has comparable biological activity.

4. The method of claim 1, wherein the PLGG1 protein:
(i) has at least 70% sequence identity to SEQ ID NO: 6;
(ii) functions as a chloroplast inner membrane glycolate/glycerate transporter and comprises the amino acid sequence set forth in SEQ ID NO: 6; or
(iii) comprises structural and functional features of the amino acid sequence of SEQ ID NO: 6 and is capable of transporting glycolate and/or glycerate across the chloroplast inner membrane.

5. The method of claim 1, wherein the reduction or loss of PLGG1 expression or activity is achieved by a technique selected from the group consisting of RNA interference (RNAi), CRISPR/Cas genome editing, TALENs, zinc finger nucleases, antisense oligonucleotides, and T-DNA insertion.

6. The method of claim 1, wherein the genetic alteration comprises introducing into the plant an RNAi construct targeting a sequence encoding the PLGG1 protein.

7. The method of claim 6, wherein the RNAi construct comprises a high percent identity to SEQ ID NO: 46.

8. The method of claim 6, wherein the RNAi construct comprises a double-stranded RNA molecule that is complementary to a portion of a polynucleotide having at least 80% sequence identity to SEQ ID NO: 46, wherein expression of said RNAi results in suppression of endogenous PLGG1 activity in the plant.

9. The method of claim 1, wherein the reduction or loss of expression of the endogenous PLGG1 protein in the C3 plant is in amount equal to or greater than the reduction or loss of expression of an endogenous tobacco (Nicotiana tabacum) PLGG1 protein in a tobacco plant comprising an RNAi construct comprising a double-stranded RNA molecule that is complementary to a portion of a polynucleotide having at least 80% sequence identity to SEQ ID NO: 46.

10. The method of claim 1, wherein the genetic alteration comprises insertion or deletion of one or more nucleotides in an open reading frame of an endogenous sequence comprising a high percent identity to SEQ ID NO: 5 over at least 16 nucleotides in length.

11. The method of claim 1, wherein the genetic alteration results in a complete loss of functional PLGG1 protein in at least a subset of chloroplasts.

12. The method of claim 1, wherein the malate synthase is at least 80% identical to amino acid residues 41-607 of SEQ ID NO: 43.

13. The method of claim 1, wherein the algal glycolate dehydrogenase is at least 80% identical to amino acid residues 41-1136 of SEQ ID NO: 45.

14. The method of claim 1, wherein the malate synthase comprises the amino acid sequence of SEQ ID NO: 43 and the glycolate dehydrogenase is an algal glycolate dehydrogenase that comprises the amino acid sequence of SEQ ID NO: 45.

15. The method of claim 1, wherein the introduction of the genetic alteration, the first heterologous polynucleotide, and the second heterologous polynucleotide:
(i) increase dry weight biomass of the plant by at least 25%;
(i) increase total starch content of the plant;
(ii) increase plant growth;
(iii) increase plant productivity;
(iv) increase light-saturated rate of photosynthesis in the plant;
(v) increase maximum carboxylation rate in the plant;
(vi) increase electron transport rate in the plant; and/or
(vii) decrease photosynthetic compensation point in the plant;
under greenhouse conditions, field conditions, or in a controlled environment compared to the C3 plant not comprising (a) and (b) of claim 1.

16. The method of claim 1, wherein the plant is selected from the group consisting of rice (Oryza sativa), corn (Zea mays), soybean (Glycine max), potato (Solanum tuberosum), cowpea (Vigna unguiculata), barley (Hordeum vulgare), wheat (Triticum aestivum), and cassava (Manihot esculenta).

17. A method of genetically altering a C3 plant, comprising introducing into a plant cell:
(a) a genetic alteration resulting in a reduction or loss of expression or activity of an endogenous sequence that encodes a chloroplast inner membrane protein; and
(b) a first heterologous polynucleotide encoding a malate synthase and a second heterologous polynucleotide encoding a glycolate dehydrogenase.

18. The method of claim 17, wherein the endogenous sequence comprises at least 80% identity to SEQ ID NO: 5 over at least 16 nucleotides in length.

19. The method of claim 17, wherein the malate synthase is at least 80% identical to amino acid residues 41-607 of SEQ ID NO: 43, and/or wherein the glycolate dehydrogenase is an algal glycolate dehydrogenase that is at least 80% identical to amino acid residues 41-1136 of SEQ ID NO: 45.

20. A genetically altered C3 plant produced by the method of claim 1.

21. A genetically altered C3 plant produced by introducing into a plant cell:
(a) a genetic alteration comprising a reduction or loss of expression or activity of an endogenous chloroplast inner membrane plastidic glycolate/glycerate translocator (PLGG1) protein in at least a portion of the chloroplasts of the C3 plant as compared to a control plant lacking the genetic alteration; and
(b) a first heterologous polynucleotide encoding a malate synthase and a second heterologous polynucleotide encoding a glycolate dehydrogenase, wherein the malate synthase and the glycolate dehydrogenase localize to a chloroplast of the plant,
wherein the quantum efficiency of photosynthesis is increased and/or the photorespiratory stress is decreased in the genetically altered C3 plant compared to a C3 plant not comprising (a) and (b).
